SEQUENCE LISTING (2 sequences)

SEQ ID NO: 1 - DNA, 22 bp, Mus musculus
agttgaggggactttcccaggc

SEQ ID NO: 2 - DNA, 27 bp, Rattus sp.
agagattgcctgacgtcagagagctag

(19) United States
(12) Patent Application Publication
(10) Pub. No.: US 2006/0040896 A1
(43) Pub. Date: Feb. 23, 2006

(54) Method and medicament for anticoagulation using a sulfated polysaccharide with enhanced anti-inflammatory activity

(75) Inventor: Thomas Preston Kennedy, Charlotte, NC (US)

Correspondence Address: ALSTON & BIRD LLP;BANK OF AMERICA PLAZA, 101 SOUTH TRYON STREET, SUITE 4000, CHARLOTTE, NC, 28280-4000, US

(73) Assignee: Paringenix, Inc.

(21) Appl. No.: 10/921,539

(22) Filed: Aug. 18, 2004

(51) Int. Cl. A61K 31/727; C08B 37/10

(52) U.S. Cl. 514/56; 536/21

(57) ABSTRACT

A method and medicament for anticoagulating a patient with a sulfated polysaccharide mixture that demonstrates enhanced anti-inflammatory activity compared to anticoagulation with unfractionated heparin comprising various combinations of fully anticoagulant unfractionated heparin with 2-O desulfated heparin demonstrating reduced anticoagulant activity but enhanced anti-inflammatory actions. The medicament preferably is administered intravenously, by aerosolization or orally. Preferably, the 2-O desulfated heparin medicament includes a physiologically acceptable carrier which may be selected from the group consisting of physiologically buffered saline, normal saline and distilled water. Additionally provided is a method of synthesizing 2-O desulfated heparin in commercially practical quantities for the formulation of an anticoagulant 2-O desulfated heparin and heparin mixture.

### BACKGROUND OF THE INVENTION

1. Field of the Invention

This invention relates to a medicament for anticoagulation using a sulfated polysaccharide with enhanced anti-inflammatory activity and to a method for treating a patient using the medicament.

2. The Prior Art

Inflammation plays a prominent role in thrombosis. With the advent of new anticoagulant strategies, enthusiastic reports have championed the anti-inflammatory benefits of blocking the coagulation cascade. In addition to retarding disseminated intravascular coagulation in septic patients through anticoagulation, activated protein C (drotecogin alfa, Xigris®) also modulates activation of the transcription factor nuclear factor-κb (NF-κB) in endothelium and monocytes (See D. E. Joyce, et al., Crit Care Med, S288-S293 (2002). More impressively, newer strategies for thrombin inhibition greatly reduce lung and renal end-organ damage in rat and primate models of sepsis (See M. S. Carraway, et al., Am J Respir Crit Care Med, Vol. 167, pp. 1200-1209 (2003). However, of the Virchow's triad of components important for thrombosis—vessel wall inflammation, blood stream stasis and hypercoagulability—there is mounting evidence that the greatest of these is inflammation, the first. Whether in the disseminated intravascular coagulation of sepsis or in coronary plaque rupture preceding acute coronary thrombosis, one of the earliest events initiating coagulation is upregulated expression of pro-coagulant events by inflammation within the vessel wall.

The simplest illustration is that of sepsis, which represents vascular inflammation in the extreme: bacterial endotoxins or exotoxins transform the endothelium from a naturally anticoagulant, pro-fibrinolytic surface to one promoting thrombosis and reduced fibrinolysis through enhanced endothelial production of the initiator of extrinsic coagulation, tissue factor (TF), forming fibrin clot, and also endothelial expression of plasminogen activator inhibitor-1 (PAI-1), which blocks endogenous dissolution of fibrin clot (See P. Libby, et al., Circulation, Vol. 103, pp. 1718-1720 (2001). The result is disseminated intravascular coagulation from activation of the extrinsic coagulation cascade, which can be partially ameliorated by the anticoagulant activity of drotecogin alpha, but even more effectively inhibited further upstream by competitive inhibition of tissue factor activity with site-inactivated Factor VIIa (See M. S. Carraway, et al., supra). A less dramatic but more prevalent example is that of arterial atherosclerosis, now uniformly recognized as a disease of chronic vascular inflammation. With progressive loss of the protective smooth muscle layer within plaques and proteolytic destruction of the fibrous plaque cap by endogenous macrophage-derived matrix metalloproteinases and neutrophil elastase (See P. K. Shah, J Am Coll Cardiol, Vol. 41, pp. 15S-22S (2003); and C. M. Dollery, et al., Circulation, Vol. 107, pp. 2829-2836 (2003), the plaque ruptures, exposing the underlying lipid-laden and macrophage-rich layers of the atherosclerotic lesion to the circulating blood stream. Many of the underlying macrophages within exposed atheroma express TF, which triggers the extrinsic coagulation cascade (See M. S. Penn, et. al., Circ Res, Vol. 89, pp 1-2 (2001). Attachment of platelets to the area of rupture can trigger platelet release of CD40 Ligand (CD40L), which interacts with CD40 on the surface of macrophages to further enhance macrophage TF production (See U. Schonbeck, et al., Circ Res, Vol. 89, pp. 1092-1103 (2001) through activation of the transcription factor nuclear factor-KB (NF-κB) (See U. Bavendiek, et al., J Biol Chem, Vol. 277, pp. 25032-25039 (2002).

Activated platelets also release P-selectin to the circulation and platelet membrane surface. P-selectin subsequently interacts with its natural ligand P-selectin glycoprotein ligand-1 (PSGL-1), present on neutrophils and monocytes, to tether circulating leukocytes to the ruptured plaque (See R. J. Shebuski, et al., J Pharmacol Exp Ther, Vol. 300, pp 729-735 (2002). CD40-CD40L- and P-selectin-mediated signaling within the milieu of the enlarging P-selectin-dependent platelet-leukocyte aggregate enhances further TF expression by leukocytes accumulating within the growing thrombus. This promotes fibrin deposition and releases TF-laden microparticles into the circulation as monocytes and neutrophils undergo apoptosis (See P. Andre, et al., Proc Natl Acad Sci USA, Vol. 97, pp. 13835-13840 (2000).

The clinical consequence of these events is presentation of patients with chest pain from the incomplete coronary occlusion of the acute coronary syndrome, or worse, total vascular thrombotic occlusion with transmural myocardial infarction. A plethora of clinical studies have now demonstrated elevation of circulating markers of inflammation in patients with stable and unstable coronary atherosclerosis, including C-reactive protein, IL-6, IL-1 receptor antagonist, TNFa, soluble adhesion molecules, including P-selectin, and other inflammatory indicators (See G. J. Blake, Curr Opin Crit Care, Vol. 9, pp. 369-374 (2003). Recent evidence suggests widespread activation of neutrophils across the coronary vascular bed, with selective transactivation of neutrophilic NF-κB and circulating platelet-leukocyte aggregates in patients with acute coronary syndromes, and even supports measurement of plasma levels of neutrophil-derived myeloperoxidase as the circulating marker of inflammation most predictive of risk for impending myocardial infarction (See A. Buffon, et al., N Engl J Med, Vol. 347, pp. 5-12 (2002; and M-L Brennan, et al., N Engl J Med, Vol. 349, pp. 1595-1604 (2003).

Vessel wall inflammation also plays a pivotal role in the genesis of deep vein thrombosis (DVT). Soluble P-selectin is thrombogenic in its own right (See P. Andre, et al., supra). P-selectin is upregulated in the vein wall in experimental DVT, and increased plasma levels of soluble P-selectin are found in patients following DVT (See D. Myers, Jr., et al., J Surg Res, Vol. 108, pp. 212-221 (2002); A. D. Blann, et al., Brit J Haematol. Vol. 108, pp. 191, 193 (2000); and L. C. Yang, et al., J Vasc Surg, Vol. 35, pp. 707-712 (2002). P-selectin deficient mice form deep vein thrombi containing significantly less fibrin (See V. V. Sullivan, et al., J Surg Res, Vol. 109, pp. 1-7 (2003). Similarly, inhibition of P-selectin with a recombinant soluble form of P-selectin glycoprotein ligand-1 (rPSGH-Ig) inhibits development of DVT in experimental models, and reduces vein wall inflammation and enhances thrombus resolution in models where DVT is already established (See M. J. Eppihimer, et al., Arterscl Throm Vasc Biol, Vol. 20, pp. 2483-2488 (2000); D. D. Myers, Jr., et al., Throm & Haemost, Vol. 85, pp. 423-429 (2001); and D. D. Myers, Jr, et al., J Vasc Surg, Vol. 36, pp 929-938 (2002).

Finally, subtle but important vessel wall inflammation may contribute to the recurring vaso-occlusive episodes characterizing sickle cell anemia. Compared to the wild type, transgenic sickle mice exhibit decreased leukocyte rolling velocity, erythrocyte microcirculatory velocity and venular blood flow rates. These differences are magnified by hypoxia-reoxygenation, when leukocyte vascular adhesion and emigration are greatly enhanced in transgenic sickle but not wild type mice. These abnormalities are abrogated by treatment of mice with antibodies to P- but not E-selectin. Plasma soluble P-selectin is elevated at baseline in patients with sickle cell disease and increases further during vaso-occlusive crises (See D. K. Kaul, et al., J Clin Invest. Vol. 106, pp. 411-420 (2000); and A. Tomer, et al., J Lab Clin Med, Vol. 137, pp. 398-407 (2001). Sickle erythrocytes adhere to immobilized recombinant P-selectin under flow conditions. Conversely, inhibition of P-selectin with an antibody, sialyl Lewis tetrasaccharide or unfractionated heparin (See N. M. Matsui, et al., Blood, Vol. 100, pp. 3790-3796 (2002); and N. M. Matsui, et al., Blood, Vol. 98, pp. 1955-1962 (2001) reduces flow adherence of sickle erythrocytes to thrombin-treated human vascular endothelium. Thus, inflammatory up-regulation of adhesion molecule expression in the vessel wall might be a root cause of morbidity and mortality in this important heritable disease.

Vascular wall inflammation has been targeted in a number of studies, using humanized murine monoclonal antibodies against ICAM-1 (CD54), the adhesion ligand for the leukocyte integrin Mac-1 (CD11b/CD18). Antibodies to ICAM-1 do not prevent lung neutrophil recruitment or injury and actually decrease survival in baboon models of sepsis, nor do they ameliorate reperfusion injury in human studies of myocardial infarction or stroke (See K. E. Welty-Wolf, et al., Am J Respir Crit Care Med, Vol. 163, pp. 665-673 (2001; D. P. Faxon, et al., J Am Coll Cardiol, Vol. 40, pp. 1199-11204 (2002); and Enlimomab acute stroke trial investigators, Neurology, Vol. 57, pp 1428-1434 (2001). The failure of this strategy may be explained by the observation that anti-ICAM-1 systemically activates complement and neutrophils, perhaps from iatrogenic creation of antigen-antibody complexes upon the vascular endothelium (See, K. Furuya, et al., Stroke, Vol. 32, pp. 2665-2674 (2001); and J. Vuorte, et al., J Immunol, Vol. 162, pp. 2353-2357 (1999). However, it is also possible that ICAM-1 is simply the wrong site to effectively block leukocyte-mediated inflammation.

While antibodies to E- and L-selectins are also ineffective, P-selectin may offer a more fruitful target. P-selectin deficient mice experience significantly decreased neointimal inflammation and remodeling after carotid angioplasty and suffer substantially less leukocyte-mediated cerebral injury in models of stroke (See, A. Kumar, et al., Circulation, Vol. 96, pp. 4333-4342 (1997); and E. S. Connolly, Jr, et al., Circ Res, Vol. 81, pp. 304-310 (1997). In addition, an antibody to E/P selectin reduces infarct volume and improves neurologic scores in a primate model of thrombotic stroke (See J. Mocco, et al., Circ Res, Vol. 91, pp. 907-914 (2002).

In targeting vascular inflammation to inhibit thrombosis, it is also possible that pharmacologic redundancy is critical. Glycoprotein IIb/IIa inhibitors offer an example. While small molecule inhibitors such as tirofiban and eptifibatide are potent selective inhibitors of this final common pathway of platelet aggregation, the humanized murine monoclonal chimeric antibody 7E3 Fab (abciximab, RheoPro®) binds and inhibits not only glycoprotein IIb/IIIa, but also the αMβ2 (Mac 1) receptor on leukocytes and the αVβ3 (vitronectin) receptor on smooth muscle and endothelium. Perhaps as a consequence of this promiscuity, abciximab also effectively suppresses the rise of systemic markers of vascular inflammation usually seen 24-48 hours after coronary angioplasty (See B. S. Coller, Am Heart J. Vol. 138, pp S1-S5 (1999); and A. M. Lincoff, et al., Circulation, Vol. 104, pp. 163-167 (2001). Thus, successful anti-inflammatory, anti-thrombotic therapies might need to block inflammatory cascades at more than one effective site in order to be successful.

Nonanticoagulant actions of heparin effectively block inflammation. In the exploration for an ideal agent with both anti-inflammatory and anti-thrombotic activities, it is possible that the search might be best directed into the past. Heparin was first discovered over 80 years ago by serendipity, when a medical student at Johns Hopkins noticed that dog liver extract prolonged plasma clotting time (See L. B. Jaques, Pharmacol Rev, Vol. 31, pp 159-166 (1979). Since then, heparin has been used in clinical medicine almost exclusively as an anticoagulant. However, heparin has numerous, redundant anti-inflammatory actions that are conferred by its polyanionic nature and are independent of its anticoagulant activity. The anti-inflammatory activities of heparin have been reviewed in detail (See L. B. Jaques, et al.; Adv Pharmacol, Vol. 46, pp 151-208 (1999); R. Lever, et al., Nature Rev Drug Disc, Vol. 1, pp 140-148 (2002); and E. Elsayed, et al., J Thromb Thrombolys, Vol. 15, pp. 11-18 (2003). Heparin has even been suggested as a fundamental construct for creating new anti-inflammatory drugs (See E. Elsayed, et al., supra.). The several anti-inflammatory activities of heparin are:

Effects on Tissue Factor. Tissue factor (TF) initiates the extrinsic coagulation cascade. Heparin potently inhibits expression of TF by endothelium, smooth muscle and monocytes in vitro, and decreases endotoxin induction of circulating TF-positive monocytes (See G. Pepe G, et al., Semin Thromb Haemost, Vol. 23, pp. 135-141 (1997); M. Attanasio, et al., Thromb Haemost. Vol. 79, pp. 959-962 (1998); F. E. Almus, et al., Blood, Vol. 77, pp. 1256-1262 (1991); Y. Cadroy, et al., Thromb Haemost, Vol. 75, pp. 190-195 (1996); J. M. Xuereb, et al., Thromb Haemost, Vol. 81, pp. 151-156 (1999); and N. Yamamoto, et al., Int J Cardiol. Vol. 75, pp 267-274 (2000). Heparin also has prominent effects on tissue factor pathway inhibitor (TFPI), which depresses the extrinsic coagulation cascade through inhibiting activities of factor VIIa and Xa. In vitro, heparin induces TFPI secretion by vascular endothelial cells and vascular smooth muscle. Heparin also directly binds to the third Kunitz domain of TFPI, thereby enhancing TFPI inhibitory activity against factors VIIa and Xa (See P. M. Sandset, et al., Haemostasis, Vol. 30 (Suppl 2), pp. 48-56 (2000); J. B. Hansen, et al., Thromb Haemost, Vol. 83, pp. 937-943 (2000); C. Lupu, et al., Arterioscleosis Thromb Vasc Biol, Vol. 19, pp. 2251-2262 (1999); U. R. Pendurthi, et al., Blood, Vol. 94, pp. 579-586 (1999); and S. Mine, et al., Biochem, Vol. 41, pp. 78-85 (2002).

In vivo heparin reduces circulating TF activity and antigen and increases TFPI levels in patients with unstable angina and myocardial infarction, and in experimental human endotoxemia (See A. M. Gori, et al., Thromb Haemost, Vol. 81, pp. 589-593 (1999); H. Soejima, et al., Thromb Res Vol. 93, pp. 17-25 (1999); N. Yamamoto, et al., Int J Cardiol, Vol. 75, pp. 267-274 (2000); and T. Pemerstorfer, et al., Circulation, Vol. 100, pp. 2485-2490 (1999). Likewise, heparin increases TPFI levels during cardiopulmonary bypass (See M. J. Adams, et al., J Cardiothor Vasc Anesth, Vol. 16, pp. 59-63 (2002); and L. B. Sun, et al., Surgery Today, Vol. 30, pp. 122-126 (2000). Heparin effects on TF and TFPI are independent of antithrombin III and represent yet another antithrombotic activity of this drug. In addition, because TF and downstream proteases of activated coagulation signal pro-inflammatory pathways, heparin-mediated inhibition of TF expression may have profound effects on amplification of inflammatory events (See M. S. Carraway, et al., supra.).

Effects on Proteases. Heparin and nonanticoagulant heparin potently inhibit neutrophil-mediated proteolytic injury by blocking the cationic neutrophil proteases cathepsin G and elastase. Heparin also blocks heparanase, which plays a prominent role in tumor cell invasion and metastases and in T-lymphocyte migration and facilitation of cellular immunity. Finally, heparin prevents induction of the matrix metalloproteinases stromelysin, gelatinase and collagenase in smooth muscle cells, an effect that might reduce the risk of fibrous cap dissolution and plaque rupture in atheromatous lesions (See A. Fryer, et al., JPharmacol Exp Ther, Vol. 282, pp 208-219 (1997); N. V. Rao, et al., Am Rev Respir Dis, Vol. 142, pp. 407-412 (1990); M. Bar-Ner, et al., Blood, Vol. 70, pp. 551-557 (1987); A. Eldor, et al., Sem Thromb Hemostas, Vol. 13, pp. 475-488 (1987); I. Vlodavskyl, et al., Adv Exper Med Biol, Vol. 233, pp. 201-210 (1988); H. P. Ekre HP, et al., Adv Exper Med Biol, Vol. 313, pp. 329-340 (1992); and R. D. Kenagy, et al., J Clin Invest, Vol. 93, pp. 1987-1993 (1994).

Effects on Inflammatory Cell Attachment and Migration. Unfractionated, but not low molecular weight, heparin blocks P-selectin, inhibits P-selectin-mediated interaction of platelets and leukocytes with blood-borne tumor cells to facilitate metastasis, and reduces P-selectin mediated adhesion of sickle erythrocytes with vascular endothelium. Through inhibition of L- and P-selectin, heparin blocks leukocyte rolling, the first step in leukocyte attachment and emigration through the vessel wall (See L. Wang, et al., J Clin Invest, Vol. 110, pp. 127-136 (2002); A. Koenig, et al., J Clin Invest, Vol. 101, pp. 877-889 (1998); L. Borsig, et al., Proc Natl Acad Sci USA, Vol. 98, pp. 3352-3357 (2001); L. Borsig, et al., Proc Natl Acad Sci USA, Vol. 99, pp. 2193-2198 (2002); and N. M. Matsui et al., Blood, Vol. 100, pp. 3790-3796 (2002). Once slowed by rolling, leukocytes more firmly affix to the vessel wall by adherence of the Mac-1 (CD11b/CD18) leukocyte integrin to ICAM-1 on the endothelial surface. Heparin blocks Mac-1 and prevents leukocyte adherence to ICAM-1. The final step in emigration is passage through the basement membrane. Proteolytic digestion of basement membrane has been proposed as a requirement for passage of leukocytes out of blood vessels, and neutrophil elastase (HLE) inhibitors block neutrophil extravasation into inflamed myocardium, bowel and lung. Through combined effects on rolling, integrin-dependent attachment and passage through the basement membrane, heparin blocks adhesion of both polymorphonuclear leukocytes and monocytes to vascular endothelium, and prevents leukocyte accumulation in areas of inflammation.

Through similar mechanism involving inhibition of heparanase, heparin also blocks lymphocyte transmigration from the vascular compartment (See M. S. Diamond, et al., J Cell Biol, Vol. 130, pp. 1473-1482 (1995); K. Peter, et al., Circulation, Vol. 100, pp. 1533-1539 (1999); C. Delclaux, et al., Am J Respir Cell Mol Biol, Vol. 14, pp. 288-295 (1996); F. A. Nicolini, et al., Am Heart J, Vol. 122, pp. 1245-1251 (1991); B. J. Zimmerman, et al., Am J Physiol, Vol. 259 (Heart Circ Physiol 28), pp. H390-H394 (1990); C. Delacourt, et al., Am JRespir Cell Mol Biol, Vol. 26, pp. 290-297 (2002); R. Lever, et al., Br J Pharmacol, Vol. 129, pp. 533-540 (2000); A. Smailbegovic, et al., Br J Pharmacol, Vol. 134, pp. 829-836 (2001); H. Jones, et al., Br J Pharmacol, Vol. 135, pp. 469-479 (2002); and I. Vlodavsky, et al., Adv Exper Med Biol, Vol. 233, pp. 201-210 (1988).

Effects on Endothelial Cell Function. Heparin decreases expression and release of the vasoconstrictor endothelin-1 by endothelia and renal mesangial cells. By this mechanism, heparin lowers blood pressure in spontaneously hypertensive rats. Heparin and nonanticoagulant heparin also preserve endothelial nitric oxide production, prevent endothelial dysfunction after ischemia-reperfusion, and enhance recovery of normal acetylcholine-induced vasorelaxation following balloon catheter injury to the arterial circulation (See K. Yokokawa, et al., J Clin Invest, Vol. 94, pp. 2080-2085 (1993); S. Reantragoon, et al., Archiv Biochem Biophys, Vol. 314, pp. 315-322 (1994); M. Kohno, et al., Kidney Intl, Vol. 45, pp. 137-142 (1994); K. Yokokawa, et al., J Am Soc Nephrol, Vol. 4, pp. 1683-1689 (1994); K. Yokokawa, et al., Am J Physiol, Vol. 263, pp. R1035-R1041 (1992); P. C. Kouretas, et al., Ann Thorac Surg, Vol. 66, pp. 1210-1215 (1998); W. C. Stembergh, 3rd, et al., J Vasc Surg, Vol. 17, pp. 318-327 (1993); P. C. Kouretas, et al., Circulation, Vol. 99, pp. 1062-1068 (1999); and J. T. Light, Jr, et al., Circulation, Vol. 88, pp. 11413-419 (1993).

Effects on Vascular Smooth Muscle and Blood Vessel Development. Heparin promotes angiogenesis and development of collateral circulation to the ischemic coronary bed, but inhibits proliferation of vascular smooth muscle, including that from human coronaries, and retards vascular restenosis following angioplasty and/or stent placement when adequate levels of drug are delivered. Heparin is also anti-proliferative for pulmonary artery smooth muscle and prevents pulmonary vascular remodeling and development of cor pulmonale in response to hypoxia (See S. M. Carroll, et al., Circulation, Vol. 99, pp. 198-207 (1993); T. C. Wright, Jr, et al., J Biol Chem, Vol. 264, pp. 1534-1542 (1989); N. M. Caplice, et al., Lancet, Vol. 344, pp. 97-98 (1994); M. A. Lovich, et al., Proc Natl Acad Sci USA, Vol. 96, pp. 11111-11116 (1999); Y. Matsumoto, et al., J Cardiovasc Pharmacol, Vol. 39, pp. 513-522 (2002); H. G. Garg, et al., Am J Physiol Lung Cell Mol Physiol, Vol. 279, pp. L779-L789 (2000); and B. T. Thompson, et al., Am J Respir Crit Care Med, Vol. 149, pp. 1512-1517 (1994).

Effects on Cytokines. Heparin avidly binds and modifies the activity of a number of cytokines and chemokines including RANTES, interleukin-8 and interleukin-6. This phenomenon was first reported for TNFa. In addition, heparin blocks secretion of IL-1, IL-6, and TNFa by monocytes, mesangial cells and intestinal epithelial cells (See A. E. Proudfoot, et al., J Biol Chem, Vol. 276, pp. 10620-10626 (2001); L. Ramdin, et al., Clin Exper Allergy, Vol. 28, pp. 616-624 (1998); R. S. Mummery, et al., J Immunol, Vol. 165, pp. 5671-5679 (2000); M. Lantz, et al., J Clin Invest, Vol. 88, pp. 2026-2031 (1991); A. Chelmonska-Soyta, et al., Comp Immunol Microbiol Inf Dis, Vol. 24, pp. 151-164 (2001); L. Cahalon, et al., Internatl Immunol, Vol. 9, pp. 1517-1522 (1997); N. M. Benador, et al., Nephron, Vol. 77, pp. 219-224 (1997); and Y. Chowers, et al., Gastroenterol, Vol. 120, pp. 449-459 (2001).

Effects on C-Reactive Protein and Complement. Heparins and nonanticoagulant heparins inhibit complement activation, enhance C-1 esterase activity, and prevent complement-mediated myocardial injury. When used therapeutically in models of myocardial infarction or in humans with inflammatory bowel disease, heparin substantially decreases circulating levels of C-reactive protein (CRP), which activates the classical complement pathway. Heparin also disrupts interaction with polycations necessary for CRP activity (See H. P. Ekre, et al., Adv Exper Med Biol, Vol. 313, pp. 329-340 (1992); J. M. Weiler, et al., J Immunol, Vol. 148, pp. 3210-3215 (1992); E. E. Calswell, et al., Archiv Biochem Biophys, Vol. 261, pp. 215-222 (1999); G. S. Friedrichs, et al., Circ Res. Vol. 75, pp. 701-710 (1994); S. C. Black, et al., Cardiovasc Res, Vol. 29, pp. 629-636 (1995); M. R. Gralinski, et al., J Pharmacol Exper Ther, Vol. 282, pp. 554-560 (1997); J. L. Park, et al., J Cardiovasc Pharmacol, Vol. 30, pp. 658-666 (1997); T. D. Barrett, et al., J Pharmacol Exper Ther, Vol. 303, pp. 1007-1013 (2002); C. Folwaczny, et al., Am J Gastroenterol, Vol. 94, pp. 1551-1555 (1999); Y. S. Ang, et al., Aliment Pharmacol Ther, Vol. 14, pp. 1015-1022 (2000); M. B. Pepys, et al., J Clin Invest, Vol. 111, pp. 1805-1812 (2003); L. A. Potempa, et al., Mol Immunol, Vol. 20, pp. 501-509 (1983); and H. Jarva, et al., J Immunol, Vol. 163, pp. 3957-3962.

Effects on Tissue Plasminogen Activation. Independent of its anticoagulant activity, heparin affects the homeostasis of normal blood fibrinolysis by several mechanisms. Bolus doses of heparin dramatically increase plasma fibrinolytic activity, elevating it as much as ten-fold in patients receiving 4 mg/kg at the initiation of cardiac bypass. This effect may be mediated largely by direct induction of endogenous tPA release from vascular endothelium. Unfractionated but not low molecular weight heparin also directly binds to tPA at a site along its kringle-2 domain. The consequent conformational change serves to enhance the plasmin-generating activity of tPA in buffers of low ionic strength, but this is not an important effect at physiological salt concentrations. Finally, heparin decreases endothelial expression and activity of plasminogen activator inhibitor-1 (PAI-1), the major tPA inhibitor normally present in plasma, and accelerates the inactivation of PAI-1 by thrombin in a mechanism that is independent of antithrombin III.

In three studies heparin 2 mg/kg was reported to produce Thrombolysis in Myocardial Infarction (TIMI) grade 2 or 3 coronary flow in as many as 52% of subjects with acute myocardial infarction, but these results were not confirmed in a recent randomized trial. However, it is possible that higher doses of heparin might be more effective at inducing a fibrinolytic state to effectively counter-balance any vascular tendency to thrombosis (See G. R. Upchurch, et al., Am J Physiol, Vol. 271, pp. H528-H534 (1996); F. Markwardt, et al., Haemostasis Vol. 6, pp. 370-374 (1977); K. Huber, et al., Thromb Res, Vol. 55, pp. 779-784 (1989); N. A. Marsh, et al., Blood Coag Fibrinolysis, Vol. 1, pp. 133-138 (1990); J. Grulich-Henn, et al., Thromb Haemost, Vol. 64, pp. 420-425 (1990); C. Vergnes, et al., Thromb Res, Vol. 63, pp. 521-530 (1991); H. P. Klocking, et al., Pharmazie, Vol. 49, pp 227-230 (1994); P. L. Stein, et al., J Biol Chem, Vol. 264, pp. 15441-15444 (1989); J. F. Liang, et al., Thromb Res, Vol. 97, pp. 349-358 (2000); P. Andrade-Gordon, et al., J Biol Chem, Vol. 264, pp. 15177-15181 (1989); T. N. Young, et al., Archiv Biochem Biophys, Vol. 296, pp. 530-538 (1992); D. C. Rijken, et al., Thromb Haemostas, Vol. 70, pp. 867-872 (1993); B. A. Konkle, et al., J Clin Invest, Vol. 82, pp 579-585 (1988); J. M. Edelberg, et al., J Biol Chem, Vol. 266, pp. 7488-7493 (1991); H. J. Ehrlich, et al., J Biol Chem, Vol. 267, pp. 11606-11611 (1992); P. A. Patston, et al., Blood, Vol. 84, pp. 1164-1172 (1994); J. C. Braga, et al., Coronary Artery Dis, Vol. 9, pp. 335-338 (1998); F. W. Verheugt, et al, J Am Coll Cardiol, Vol 31, pp. 289-293 (1998); S. K. Dwivedi, et al., Indian Heart J, Vol. 52, pp 183-186 (2000); and A. Liem, et al., J Am Coll Cardiol, Vol. 35, pp. 600-604 (2000).

Heparin's unique and redundant combination of anti-inflammatory activities has been applied in limited fashion to treat a number of important diseases. In humans, heparin blunts endotoxin-induced coagulation activation, alleviates myocardial ischemia from Kawasaki disease, reduces colonic inflammation in severe active ulcerative colitis as effectively as corticosteroids, and substantially decreases rates of crises and hospitalization in patients with sickle cell disease. Of intriguing interest is a randomized, controlled chronic trial of heparin in the prevention of cardiovascular disease. Subjects with prior myocardial infarction were randomized to receive 30,000 units of subcutaneous heparin twice weekly (n=105) or placebo (n=117) for two years. There were 21 cardiovascular deaths and 18 non-fatal cardiovascular events (myocardial infarction or stroke) in the placebo group, but only 4 deaths and 5 non-fatal events in patients receiving heparin (P<0.01). The large difference between groups (39 deaths and events for placebo but only 9 for heparin) could not be explained by anticoagulation, since therapeutic anticoagulation lasted less than a day following each heparin injection. Conducted in 1956, this study has never been repeated.

One important limiting factor in maximizing anti-inflammatory effects of heparin in the clinical arena has been the risk of therapeutic anticoagulation. In animal models, however, when administered in larger than anticoagulant doses, heparin and nonanticoagulant heparins substantially reduce leukocyte-mediated ischemia-reperfusion injury in models of myocardial infarction, stroke and hepatic and renal failure. Thus, the development of a non-anticoagulant heparin that retains anti-inflammatory actions will enable improved treatment of diseases with both procoagulant and proinflammatory components, including acute coronary syndromes, sepsis, and acute lung injury (See T. Pemerstorfer, et al., supra; G. S. Friedrichs, et al., supra; S. C. Black, et al., supra; V. H. Thourani, et al., Am JPhysiol Heart Circ Physiol, Vol. 48, pp. H2084-H2093 (2000); S. Tateno, et al., Circulation, Vol. 103, pp. 2591-2597 (2001); C. Folwaczny, et al., Am J Gastroenterol, Vol. 94, pp 1551-1555 (1999); Y. S. Ang, et al., Aliment Pharmacol Ther, Vol. 14, pp 1015-1022 (2000); H. Chaplin, Jr, et al., E African Med J, Vol. 66, pp. 574-584 (1989); H. Engelberg, et al., Circulation, Vol. 13, pp. 489-498 (1956); K. Yanaka, et al., J Neurosurg, Vol. 85, pp. 1102-1107 (1996); K. Yanaka, et al., J Neurosurg, Vol. 85, pp. 1108-1112 (1996); and T. Zhou, et al., World J Gastroenterol, Vol. 8, pp. 897-900 (2002).

### SUMMARY OF THE INVENTION

It is an object of the present invention to provide a method for producing a heparin product that functions as an anticoagulant and antithrombotic.

It is another object of this invention to provide a heparin product that is sufficiently large enough in size and possessing of sufficient degree of retained sulfation, so that the heparin product is not only an anticoagulant for the blood, but also has greatly enhanced anti-inflammatory activity compared to currently available unfractionated heparin.

It is an object of the present invention that the therapeutic agent is produced from a toxicologically characterized compound.

Another object of this invention is that the synthesis of the 2-O desulfated heparin contained in this product can be produced at commercially feasibly levels using a simple process.

Consideration of the specification, including the several figures and examples to follow will enable one skilled in the art to determine additional objects and advantages of the invention.

The present invention provides a heparin medicament that is equally anticoagulant compared to unfractionated heparin, but has greatly enhanced anti-inflammatory activity, comprising a treatment effective amount of 2-O desulfated heparin mixed with unfractionated heparin in a physiologically acceptable carrier. The physiologically acceptable carrier may be selected from the group consisting essentially of physiologically buffered saline, normal saline and distilled water. The invention also provides a medicament comprising a dose of between 3 mg/kg patient body weight and 100 mg/kg, but preferably 3.5-25 mg/kg. These doses are also provided in a physiologically acceptable carrier. In preferred embodiments of the invention the product mixture of 2-O desulfated heparin and unfractionated heparin can be administered by aerosolization, by intravenous injection, by subcutaneous injection, or orally. An effective dose for administration to a human, especially when used intravenously, is a dose between 3 mg/kg and 100 mg/kg of the mixture of 2-O desulfated heparin and unfractionated heparin.

In other embodiments of the invention, the product is produced by mixing portions of 2-O desulfated heparin and unfractionated heparin in ratios of 5 to 20 parts 2-0 desulfated heparin to 0 to 1 part of unfractionated heparin (weight to weight). In still other preferred embodiments of the invention, the 2-O desulfated heparin is produced from porcine intestinal heparin and mixed with unfractionated porcine intestinal heparin to produce the anticoagulant heparin with enhanced anti-inflammatory activities. Preferably, the medicament includes a physiologically acceptable carrier which may be selected from the group consisting of physiologically buffered saline, normal saline, and distilled water. The present invention further provides a method of producing an anticoagulant heparin product with enhanced anti-inflammatory properties comprising reducing heparin in solution and lyophilizing the reducing heparin solution.

In another embodiment, the anticoagulant heparin product with substantially enhanced anti-inflammatory properties is produced by lyophilizing heparin in solution without reducing it. In a preferred embodiment, the pH of the reduced or non-reduced heparin solution is raised above 13.

### BRIEF DESCRIPTION OF THE DRAWINGS

The foregoing and other objects, advantages and featues of the invention, and manners in which the same are accomplished, will become apparent from the following detailed description of the inventon taken in conjunction with the accompanying drawings which illustrate preferred and exemplary embodiments, wherein:

FIG. 1 shows a chemical formula of the pentasaccharide binding sequence of unfractionated heparin and the comparable sequence of 2-O, 3-O desulfated heparin (ODS Heparin);

FIG. 2 shows the differential molecular weight distribution plots determined by multiangle laser light scattering, in conjunction with high performance size exclusion chromatography, of ODS Heparin compared to the parent porcine intestinal heparin from which it was produced;

FIG. 3 shows disaccharide analysis of heparin and the 2-O, 3-O desulfated heparin (ODS heparin) of this invention;

FIG. 4 shows a proposed reaction scheme for desulfating the 2-O position of a-L-iduronic acid in the pentasaccharide binding sequence of heparin;

FIG. 5 shows a graph of the hemoglobin content measured in the bronchoalveolar lavage fluid 24 hours after administration of saline (control), human leukocyte elastase (HLE), HLE plus heparin, and HLE plus ODS heparin;

FIG. 6 shows a graph of the concentration of protein in the bronchoalveolar lavage fluid 24 hours after administration of saline (control), human leukocyte elastase (HLE), HLE plus heparin, and HLE plus ODS heparin;

FIG. 7 shows a graph of the number of polymorphonuclear leukocyte (PMN) cells in the bronchoalveolar lavage fluid 24 hours after administration of saline (control), human leukocyte elastase (HLE), HLE plus heparin, and HLE plus ODS heparin;

FIG. 8 is a graph showing that heparin and ODS desulfated heparin reduce plasma infarct size (ratio of area necrosis/area at risk, or AN/AAR);

FIG. 9 demonstrates that heparin and ODS heparin reduce plasma creatine kinase activity after myocardial infarction;

FIG. 10 demonstrates that heparin and ODS heparin reduce influx of polymorphonuclear leukocytes (PMNs) into myocardium after myocardial infarction, measured by the activity of the PMN specific enzyme myeloperoxidase in myocardial tissue;

FIG. 11 shows that ODS heparin does not produce anticoagulation in vivo, measured by the activated clotting time (ACT), but that identical amounts of heparin produce profound anticoagulation, measured by prolongation of the ACT;

FIG. 12 demonstrates that heparin and ODS heparin block PMN adherence to normal coronary artery endothelium in vitro;

FIG. 13 illustrates that heparin and ODS heparin reduce PMN adherence to post-experimental coronary artery endothelium;

FIG. 14 shows that heparin and ODS heparin preserve the vasodilator function of ischemic-reperfused coronary arteries;

FIG. 15A demonstrates that nuclear factor-κB (NF-κB, brown stained) is normally present in the cytoplasm of unstimulated human umbilical vein endothelial cells (HUVECs);

FIG. 15B shows that HUVECs stimulated with tumor necrosis factor a (TNFa) without addition of heparin. Some, but not all nuclei now stain positive for anti-p65, corresponding to trans.

FIG. 15C shows that TNFa stimulation fails to produce translocation of NF-κB from cytoplasm to the nucleus in HUVECs pre-treated with 200 μg/mL ODS heparin;

FIG. 16 are electrophoretic mobility shift assays of nuclear protein showing that ODS heparin decreases NF-κB DNA binding in TNF-stimulated HUVECs;

FIG. 17 are electrophoretic mobility shift assays of nuclear protein from ischemic-reperfused rat myocardium showing that ODS heparin decreases NF-κB DNA binding stimulated by ischemia-reperfusion;

FIG. 18 shows mean values of activated partial thromboplastin time (APTT) at baseline and 0.25, 1, 4, and 8 hours in dogs treated with 0, 4, 12 and 24 mg/kg of 2-O desulfated heparin;

FIG. 19 shows mean values of area at risk (AAR) as a percentage of left ventricle mass (LV) for the closed chest pig infarction model treated with 0, 5, 15 or 45 mg/kg 2-O desulfated heparin; and

FIG. 20 shows mean values of infarct size, expressed as area of necrosis (AN) as a percentage of area at risk (AAR), for the closed chest pig infarction model treated with 0, 5, 15 or 45 mg/kg 2-O desulfated heparin.

### DETAILED DESCRIPTION OF THE INVENTION

The present invention will now describe more fully hereinafter with reference to the accompanying examples, in which preferred embodiments of the invention are shown. This invention, may, however, be embodied in many different forms and should not be construed as limited to the embodiments set forth herein; rather these embodiments are provided so that this disclosure will be thorough and complete, and will fully convey the scope of the invention to those skilled in the art.

As noted, unfractionated heparin has multiple anti-inflammatory activities. However, at normal blood levels of heparin required for appropriate anticoagulation of the bloodstream [loading dose of about 80 U/kg (0.5 mg/kg), followed by about 12 U/kg/hour (0.1 mg/kg/hour as an infusion], the magnitude of this anti-inflammatory activity is limited and is at the low end of the dose-response curve for anti-inflammatory actions of unfractionated heparin (See A. Koenig, et al., J Clin Invest, Vol. 101, pp. 877-889 (1998). Higher doses of unfractionated heparin in the range of 3 to 10 mg/kg are required for maximal anti-inflammatory effects, but it is not possible to administer these doses without increasing the level of anticoagulant effect measured by activated partial thromboplastin time (APTT) to as much as 600 seconds, which is 10-fold higher than that which would be desirable for therapeutic anticoagulation (See S. C. Black, et al., supra.).)

It has also been found that a variety of nonanticoagulant heparins (N-desulfated; 2-O, 3-O or 6-O desulfated; N-desulfated and reacetylated; and O-decarboxylated heparin) can attenuate inflammatory responses in vivo, such as inhibiting the destructive effects of human leukocyte elastase (HLE) on lung when instilled in the trachea. These same heparins and nonanticoagulant heparins can attenuate ischemia-reperfusion injury in the heart, brain and other organs and reduce the size of organ infarction as measured by the size of organ necrosis. Examples of the preparation of 2-O desulfated nonanticoagulant heparin, which is also 3-O desulfated, may be found in, for example, U.S. Pat. No. 5,668,188; U.S. Pat. No. 5,912,237; and U.S. Pat. No. 6,489,311, incorporated herein by reference. The amounts of 2-O desulfated heparin may be given in amounts of 3 mg/kg to 100 mg/kg, but preferably in amounts from about 3.5 mg/kg to 25 mg/kg.

The nonanticoagulant heparin 2-O desulfated heparin has the advantage of inhibiting inflammation such as HLE-induced lung inflammation or myocardial inflammation induced by ischemia-reperfusion, but without the side effect of excessive anticoagulation that would result from equivalent doses of unmodified heparin. Low molecular weight heparins (See K. Yanaka, et al., supra.) at doses of approximately 9 mg/kg inhibit inflammation, but this greatly exceeds their usual anticoagulant dose of 1 mg/kg, leading to excessive anticoagulation that is long-lived in effect for 24 hours or even greater. Other sulfated polysaccharide heparin analogs (See K. S. Kilgore, et al., J Pharmacol Exp Therap, Vol. 285, pp. 987-994 (1998) can also inhibit inflammation, but lack functional anticoagulant activity.

The purpose of the present invention to disclose a method for producing an anticoagulant heparin product which is fully anticoagulant but also exhibits greatly enhanced anti-inflammatory pharmacology compared to unfractioned heparin at the level of therapeutically appropriate anticoagulation. This anti-inflammatory anticoagulant is a mixture of 2-O desulfated heparin, which has greatly reduced anticoagulant activity, with sufficient amounts of fully anticoagulant unfractionated heparin to render the combination effective as an anticoagulant but demonstrating greatly enhanced anti-inflammatory pharmacology.

The partially desulfated heparin 2-O desulfated heparin preferred for manufacture of this mixture is produced as outlined in U.S. Pat. No. 5,668,188; U.S. Pat. No. 5,912,237; and U.S. Pat. No. 6,489,311, and incorporated herein by reference, by reducing heparin in solution and drying, lyophilizing or vacuum distilling the reduced heparin solution. The starting heparin is placed in, for example, water or other solvent. The typical concentration of the heparin solution can be from 1 to 10 percent heparin. The heparin used in the reaction can be obtained from numerous sources, known in the art, such as porcine intestine or beef lung. The heparin that has been modified is used in any number of ways known to those of skill in the art, such as lower molecular weight heparins produced by periodate oxidation or nitrous acid depolymerization. One can also use as starting material the currently available fully anticoagulant low molecular weight heparins enoxaprin or dalteparin. Many other possible starting materials will be apparent to those of skill in the art, given the teaching provided herein.

The selected heparin starting material in solution can be reduced in solution by incubating it with a reducing agent, such as sodium borohydride, catalytic hydrogen, or lithium aluminum hydride. A preferred reduction of heparin is performed by incubating the heparin with sodium borohydride, typically at a concentration (wt/vol) of 1%, or 10 grams of NaBH₄ per liter of solution. Additionally, other known reducing agents can be utilized. The incubation with reducing substance can be achieved over a wide range of temperatures, taking care that the temperature is not so high that the heparin caramelizes. A suggested temperature range is about 4° C. to 30° C., or even about 20° C. to about 25° C. The length of the incubation can also vary over a wide range as long as it is sufficient for reduction to occur. For example, several hours to overnight (i.e., about 4 to about 12 hours) can be sufficient. However, the time can be extended to over several days, for example, exceeding about 60 hours. Alternatively to reduction of the heparin, which preserves its molecular weight during lyophilization, one can omit this step and proceed directly to lyophilization or drying for production. However, depolymerization will occur more intensely without the reducing step and the molecular weight of the resulting product will be predictably lower.

The anti-inflammatory activity of 2-O desulfated heparin occurs over a wide dosing range, and begins at 1.5 mg/kg, increasing up to 45 mg/kg. Thus, the mixing ratio (weight/weight) of unfractionated anticoagulant heparin to 2-O desulfated heparin is precisely adjusted to produce a series of fully anticoagulant heparins with either slightly enhanced anti-inflammatory activity (low 2-O desulfated heparin content) ranging to those with greatly enhanced anti-inflammatory actions (high 2-O desulfated heparin content). An example of a heparin mixture with modestly enhanced anti-inflammatory actions, is a mixture of 4.5 kg of 2-O desulfated heparin with 0.5 kg of USP unfractionated porcine intestinal heparin. To achieve both therapeutic anticoagulation with simultaneous anti-inflammatory actions, the resulting mixture can be administered to a patient at an intravenous loading dose of 5 mg/kg, followed by an infusion of about 1.0 to 1.5 mg/kg/hour, adjusted upward or downward to achieve a therapeutic APTT of 2½ times control, or of about 50-80 seconds. For even greater anti-inflammatory actions with full anticoagulation, the ratio of 2-O desulfated heparin to unfractionated USP heparin can be increased to a mixture of 9.5 kg 2-O desulfated heparin to 0.5 kg of unfractionated USP heparin. This mixture is administered at a loading dose of 10 mg/kg intravenously, followed by an infusion rate of about 0.9 to 2.5 mg/kg/hour, adjusted upward or downward to achieve a therapeutic APTT of 2½ time control, or of about 50-80 seconds.

For near maximal anti-inflammatory actions with therapeutic anticoagulation, the unfractionated heparin can even be omitted entirely. Under these circumstances, to achieve near maximal anti-inflammatory activity at therapeutic levels of anticoagulation, the unfractionated heparin is omitted entirely, and 2-O desulfated heparin is given in an intravenous loading dose of 12-15 mg/kg, followed by an infusion rate of 0.7 to 3.0 mg/kg/hour, adjusted upward or downward to achieve an APTT of 50-80 seconds. Even larger intravenous doses of 2-O desulfated heparin (up to 45 mg/kg) can be administered alone to achieve supramaximal levels of anticoagulation, along with greatly enhanced anti-inflammatory effects, under conditions such as cardiopulmonary bypass or cardiac catherization procedures, where the therapeutic goal of anticoagulation is much higher. Under these circumstances, the degree of anticoagulation can be monitored by measuring the activated clotting time (ACT) to achieve prolongation to 300-400 seconds. It is noted that the rate of infusion to maintain a constant drug level diminishes with greater bolus, due to decreased clearance of drug following higher bolus levels, demonstrated in Table I of Example XIII.

Additionally, the preferred method for producing 2-O desulfated heparin further comprises raising the pH of the reduced or unreduced heparin to 13 or greater by adding a base capable of raising the pH to 13 or greater to the reduced or non-reduced heparin solution. The pH can be raised by adding any of a number of agents including hydroxides, such as sodium, potassium or barium hydroxide. A preferred agent is sodium hydroxide (NaOH). Even once a pH of 13 or greater is achieved, it can be beneficial to further increase the concentration of the base. For example, it is preferable to add NaOH to a concentration of about 0.25 M to about 0.5 M NaOH. This alkaline solution is then dried, lyophilized or vacuum distilled.

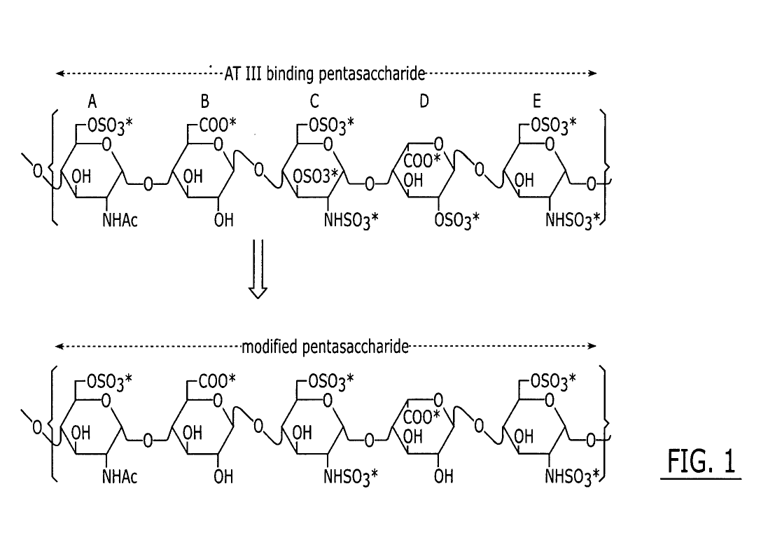

The partially desulfated heparin produced by such methods as outlined in U.S. Pat. No. 5,668,188; U.S. Pat. No. 5,912,237; and U.S. Pat. No. 6,489,311, is a 2-O desulfated heparin that is also largely 3-O desulfated and possesses a degree of sulfation of approximately 1.0 (5 sulfate groups per pentasaccharides; see FIG. 1). If unfractionated porcine heparin with an average molecular weight of 11.5 kD is used as a starting material and this is reduced with sodium borohydride prior to lyophilization, the resulting product has an average molecular weight of 10.5 kD. Preferably the starting material is unfractionated porcine intestinal heparin, but it can also be unfractionated bovine lung heparin. Preferably, 2-O desulfated heparin is employed as the mixture component with reduced anticoagulant activity. However, other heparin derivatives with reduced anticoagulant activity may be employed, including N-desulfated heparin, periodate-oxidized heparins, 6-O desulfated heparin and carboxylate reduced heparins, mixing in the proper ratio of unmodified, fully anticoagulant porcine or bovine heparin so as to result in a mixed product with both full anticoagulant and antithrombotic activities and greatly enhanced anti-inflammatory actions. Methods for manufacture of these other non-anticoagulant heparin entities are known in the art.

Lower molecular weight heparins such as produced by controlled nitrous acid depolymerization, alkaline degradation of heparin benzyl ester, or other methods known in the art can be used as starting materials, resulting after alkaline lyophilization in low-molecular weight heparin derivatives with greatly reduced anti-coagulant activity. These low molecular weight reduced anticoagulant heparins can then be mixed with fully anticoagulant low molecular weight heparin starting material in precise portions to result in a low molecular weight heparin with greatly enhanced anti-inflammatory activity, yet typical low molecular weight heparin pharmacokinetics, including predictable vascular absorption after subcutaneous injection, predicatably high sustained blood levels and long vascular half-lives. Commercially available low molecular weight heparins such as enoxaprin or dalteparin provide readily available staring materials. For example, beginning with enoxaprin sodium, the alkaline lyopilization product of this low molecular weight heparin might be mixed in a ratio of 5 to 15 mg of alkaline lyophilized enoxaprin to 1 mg of enoxaprin starting material. This mixture would then be administered in a dose of 6 mg/kg to 16 kg/kg, respectively, at a frequency of every 12 hours to provide both therapeutic anticoagulation and reduction of inflammation. Similar ratios and mixtures of dalteparin and its alkaline lyophilized product would be used to the same end. Using low molecular weight heparins as starting materials to make 2-O desulfated heparin and mix with it thereafter, the level of anticoagulation can be monitored by following anti-factor Xa activity.

The medicaments of this invention are useful for simultaneously anticoagulating a patient and treating inflammation in adult respiratory distress syndrome, ischemia-reperfusion syndromes, myocardial infarction, stroke, neurologic transient ischemic attacks, atherosclerosis, atherosclerotic vascular disease, acute coronary syndromes, diabetic vascular disease, sepsis, septic shock, disseminated intravascular coagulation, pulmonary embolism, deep vein thrombosis, inflammatory bowel disease, ulcerative colitis, portal vein thrombosis, renal vein thrombosis, thrombosis of the brain venous sinuses, glomerulonephritis, wounds, sickle cell disease, or cutaneous burns.

Depending upon the intended mode of administration, the pharmaceutical compositions may be in the form of a solid, semi-solid or liquid dosage forms, such as, for example, tablets, suppositories, pills, capsules, powders, liquids, suspensions, lotions, creams, gels, or the like, preferably in unit dosage form suitable for single administration of a precise dosage. The compositions will include an effective amount of the selected drug in combination with a pharmaceutically acceptable carrier and, in addition, may include other medicinal agents, pharmaceutical agents, carriers, adjuvants, diluents, etc.

The invention additionally provides aerosol particles comprising a physiologically acceptable carrier and an effect amount of 2-O desulfated heparin or analog thereof. The particles can consist of essentially of particles less than 10 microns and preferably less than 5 microns. For delivery to the airway or lung, 2-O desulfated heparin can be delivered as a micronized power or inhaled as a solution with the use of a commercially available nebulizer device. For delivery to the nasal mucosa, 2-O desulfated heparin can be administered as a solution that is aerolized by a commercially available misting or spray device, or it can be delivered as a nasally administered micronized dry powder.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmaceutically administrable compositions can, for example, be prepared by dissolving, dispersing, etc. an active compound as described herein and optional pharmaceutical adjuvants in an excipient, such as, for example, water, saline, aqueous dextrose, glycerol, ethanol, and the like, to thereby form a solution or suspension. If desired, the pharmaceutical composition to be administered may also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, etc. Liquid compositions can be aerosolized for administration. Actual methods of preparing such dosage forms are known, or will be apparent, to those skilled in this art; for example, see Remington's Pharmaceutical Sciences, E. W. Martin (ed.), Mack Publishing Co., Easton, Pa.

For oral administration, fine powders or granules may contain diluting, dispersing, and/or surface active agents, and may be presented in water or in a syrup, in capsules or sachets in the dry state, or in a non-aqueous solution or suspension wherein suspending agents may be included, in tablets wherein binders and lubricants may be included, or in a suspension in water or a syrup. Where desirable or necessary, flavoring, preserving, suspending, thickening, or emulsifying agents may be included. Tablets and granules are preferred oral administration forms, and these may be coated. To enhance oral penetration and gastrointestinal absorption, 2-O heparin can be formulated with mixtures of olive oil, bile salts, or sodium N-[8-(2 hydroxybenzoyl)amino] caprylate (SNAC). A preferable ratio of about 2.25 g of SNAC to 200 to 1,000 mg 2-O desulfated heparin is employed. Additional formulations that facilitate gastrointestinal absorption can be made by formulating phospholipids-cation precipitate cochleate delivery vesicles of 2-O desulfated heparin with phosphotidylserine and calcium, using methods described in U.S. Pat. Nos. 6,153,217; 5,994,318; 5,840,707, among others.

For rectal administration, 2-O desulfated heparin can be administered in a suppository, foam, gel, solution or enema.

Parenteral administration, if used, is generally characterized by injection. Injectables can be prepared in conventional forms, either as liquid solutions or suspensions, solid forms suitable for solution or suspension in liquid prior to injection, or as emulsions. A more recently revised approach for parenteral administration involves use of a slow release or sustained release system, such that a constant level of dosage is maintained. See, e.g., U.S. Pat. No. 3,710,795, which is incorporated by reference herein.

By “pharmaceutically acceptable” is meant a material that is not biologically or otherwise undesirable, i.e., the material may be administered to an individual along with the 2-O, 3-O desulfated heparin or heparin analog without causing any undesirable biological effects or interacting in a deleterious manner with any of the other components of the pharmaceutical composition in which it is contained.

The present invention is more particularly described in the following examples which are intended as illustrative only since numerous modifications and variations therein will be apparent to those skilled in the art.

### EXAMPLE I

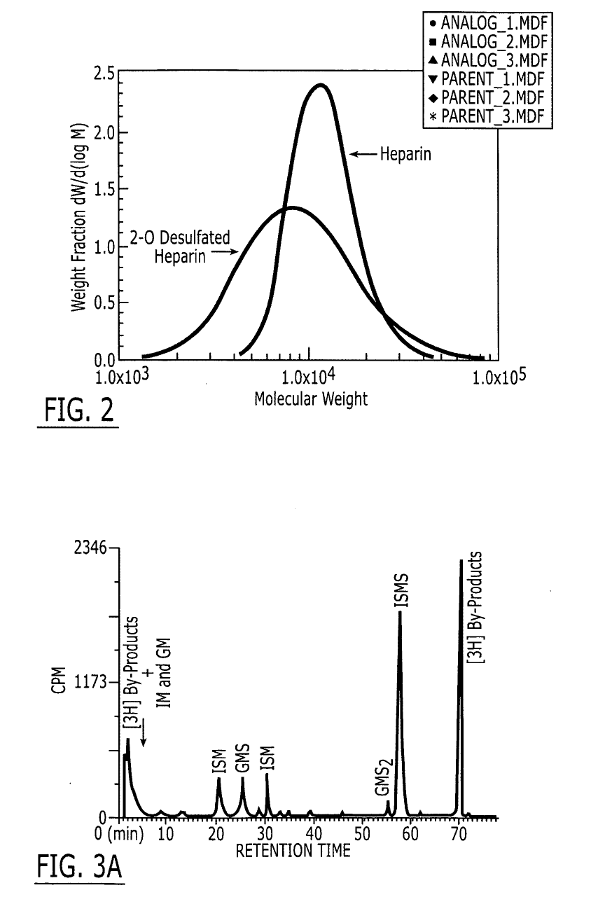

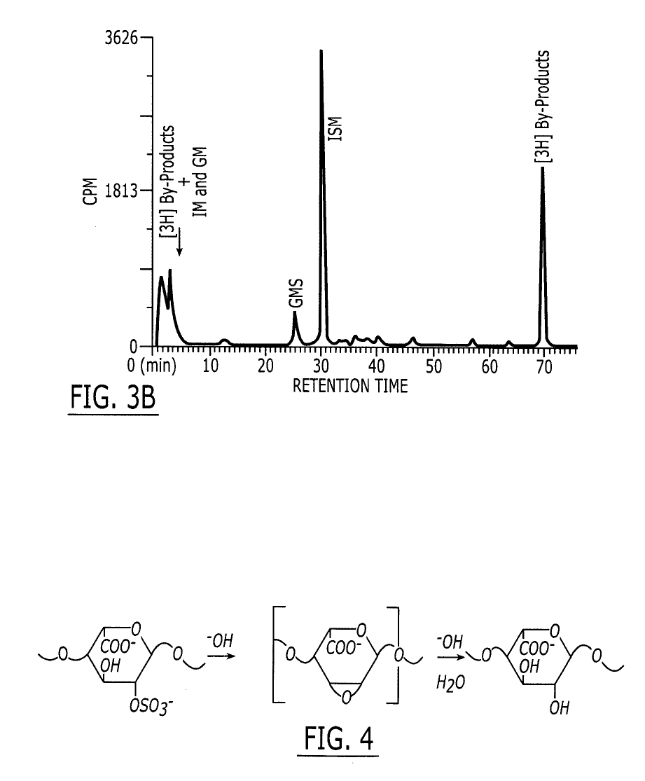

Production of 2-O Desulfated Heparin with Reduced Anticoagulant Activity. Partially desulfated 2-O desulfated heparin can be produced in commercially practical quantities by methods described above in U.S. Pat. No. 5,668,188; U.S. Pat. No. 5,912,237; and U.S. Pat. No. 6,489,311. Heparin modification was made by adding 500 gm of porcine intestinal mucosal sodium heparin to 10 L deionized water (5% final heparin concentration). Sodium borohydride was added to 1% final concentration and the mixture was incubated overnight at 25° C. Sodium hydroxide was then added to 0.4 M final concentration (pH greater than 13) and the mixture was lyophilized to dryness. Excess sodium borohydride and sodium hydroxide were removed by ultrafiltration. The final product was adjusted to pH 7.0, precipitated by addition of three volumes of cold ethanol and dried. The 2-O desulfated heparin produced by this procedure was a fine crystalline slightly off-white powder with less than 10 USP units/mg anticoagulant activity and less than 10 anti Xa units/mg anticoagulant activity. The structure of this heparin is shown in FIG. 1. Molecular weight was determined by high performance size exclusion chromatography in conjunction with multiangle laser light scattering, using a miniDAWN detector (Wyatt Technology Corporation, Santa Barbara, Calif.) operating at 690 nm. Compared with an average molecular weight of 13.1 kD for the starting material, the 2-O desulfated heparin had an average molecular weight of 11.8 kD. Demonstrated in FIG. 2 are the differential molecular weight distributions of the parent molecule and the 2-O desulfated heparin. Disaccharide analysis was performed by the method of Guo, et al. (Guo Y, et al., Analysis of oligosaccharides from heparin by reversed-phase ion-pairing high-performance liquid chromatography. Anal Biochem 178:54-62, 1988). Compared to the starting material shown in FIG. 3A, the 2-O desulfated heparin shown in FIG. 3B is characterized by conversion of ISM [L-iduronic acid(2-sulfate)-2,5-anhydromannitol] to IM [L-iduronic acid-2,5-anhydromannitol], and ISMS [L-iduronic acid(2-sulfate)-2,5 anhydromannitol(6-sulfate)] to IMS L-iduronic acid-2,5-anhydromannitol(6-sulfate), both indicating 2-O desulfation. The proposed sequence of 2-O desulfation is shown in FIG. 4. The heparin modified as described above also includes a 3-O desulfated heparin, characterized by conversion of GMS2 [D-glucuronic acid-2,5-anhydromannitol(3,6-disulfate)] to GMS [D-glucuronic acid-2,5-anhydromannitol(6-sulfate)], indicating 3-O desulfation.

### EXAMPLE II

Production of 2-O Desulfated Heparin with Reduced Anticoagulant Activity and Inhibitory Activity for Human Leukocyte Elastase. USP porcine intestinal heparin is purchased from a reliable commercial vendor such as Scientific Protein Laboratories (SPL), Wanaukee, WI. It is dissolved at room temperature (20±5° C.) to make a 5% (weight/volume) solution in deionized water. As a reducing step, 1% (weight/volume) sodium borohydride is added and agitated for 2 hours. The solution is then allowed to stand at room temperature for 15 hours. The pH of the solution is then alkalinized to greater than 13 by addition of 50% sodium hydroxide. The alkalinized solution is agitated for 2-3 hours. This alkalinized solution is then loaded onto the trays of a commercial lyophilizer and frozen by cooling to −40° C. A vacuum is applied to the lyophilizer and the frozen solution is lyophilized to dryness. The lyophilized product is dissolved in cold (<10° C.) water to achieve a 5% solution. The pH is adjusted to about 6.0 by slow addition of hydrochloric acid with stirring, taking care to prevent the solution temperature at <15° C. The solution is then dialyzed with at least 10 volumes of water or subjected to ultrafiltration to remove excess salts and reducing agent. To the dialyzed solution, an amount of 2% sodium chloride (weight/volume) is added. The 2-O desulfated heparin product is then precipitated using one volume of hysol (denatured ethanol). After the precipitation has settled for about 16 hours, the supernatant is siphoned off. The precipitate is re-dissolved in water to a 10% (weight/volume) solution. The pH is adjusted to 5-6 using hydrochloric acid or sodium hydroxide, the solution is filtered through a 0.2μ filter capsule into a clean container. The filtered solution is then lyophilized to dryness. The resulting product can be made by this method with yields up to 1.5 kg. The final product is a 2-O desulfated heparin with a pH of 6.4, a USP anticoagulant activity of about 6 U/mg, and an anti-Xa anticoagulant activity of 1.9 U/mg. The product is free of microbial and endotoxin contamination, and the boron content measured by ICP-AES is <5 ppm. This 2-O desulfated heparin has been tested for in rats and dogs at doses as high as 160 mg/kg daily for up to 10 days, with no substantial toxicity.

The resulting 2-O desulfated heparin is useful for inhibiting the enzymatic activity of human leukocyte elastase. This is tested by methods detailed in U.S. Pat. No. 5,668,188; U.S. Pat. No. 5,912,237; and U.S. Pat. No. 6,489,311, incorporated herein by reference. Briefly, the inhibition of human leukocyte elastase (HLE) was measured by incubating a constant amount of HLE (100 pmol) with a equimolar amount of 2-O desulfated heparin (I/E ratio 1:1) for 30 minutes at 25 C in 500 μL of Hepes buffer (0.125 M, 0.125% Triton X-100, pH 7.5) diluted to the final volume of 900 μL. The remaining enzyme activity is measured by adding 100 μL of 3 mM N-Suc-Ala-Ala-Val-nitroanalide (Sigma Chemical, St. Louis, Mo., made in dimethylsulfoxide). The rate of change in absorbance of the proteolytically released chromogen 4-nitroanline is monitored at 405 nm. The percentage inhibition is calculated based upon enzyme activity without inhibitor. The 2-O desulfated heparin produced by above methods inhibits HLE >90% at a 1:1 enzyme to inhibitor molar ratio.

The bulk product can be formulated into convenient unit dose vials of 50 mg/ml. This is accomplished by adding 2-O desulfated heparin to USP sterile water for injection to make a 6.5% (weight/weight) solution. Sodium chloride and sterile water for injection are added to adjust the final osmolality to 280-300 mOsm, and the pH is adjusted to 7.1-7.3 using 1 N hydrochloric acid or sodium hydroxide, as needed. The solution is filtered and transferred to a sterile fill Class 100 area where unit dose glass vials are filled with 21 ml solution each, sealed, crimped and labeled.

### EXAMPLE III

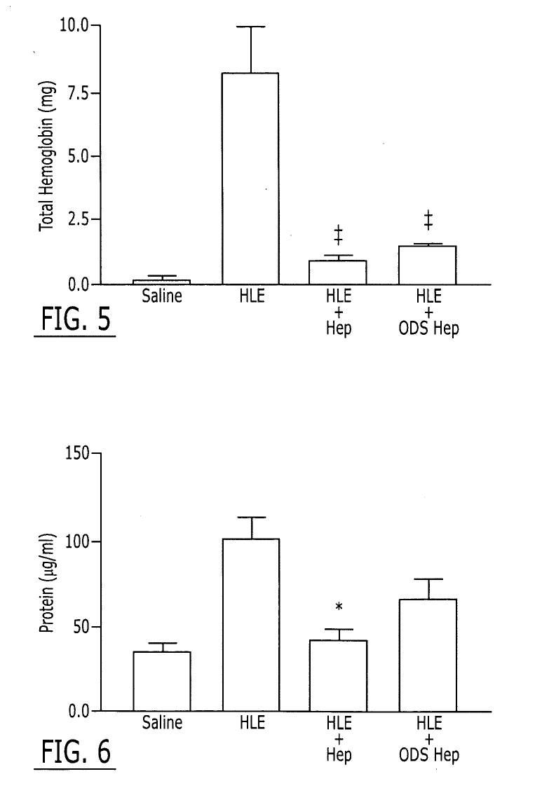

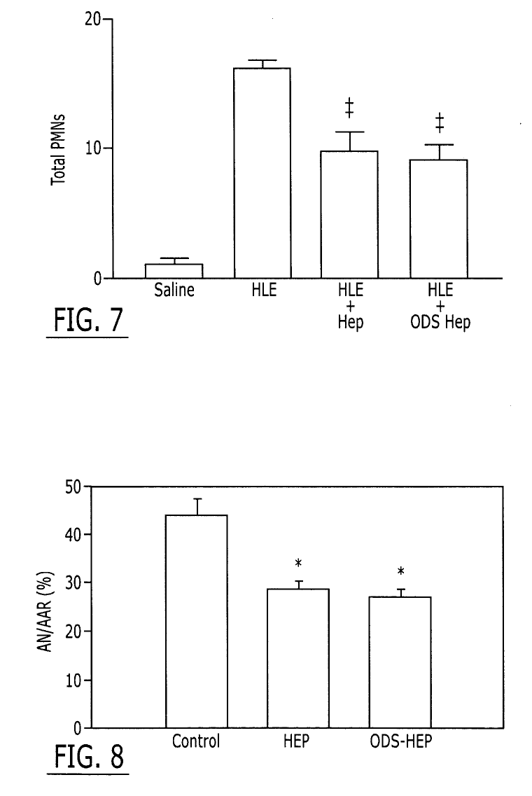

Prevention of Lung Injury from Human Leukocyte Elastase with 2-O Desulfated Heparin. The ability of 2-O desulfated heparin to prevent human leukocyte elastase (HLE)-mediated lung injury was assessed in female golden Syrian hamsters (Harlan Industries, Indianapolis, Ind.) weighing 90 to 110 g. Phenobarbital-anesthetized hamsters were injected intratracheally with 0.25 ml sterile 0.9% saline (NS), 0.25 ml NS containing HLE (100 μg) or 0.25 ml NS containing 500 μg of heparin (Sigma) or 2-O desulfated heparin according to Example I followed by 0.25 ml NS with HLE. Animals were killed by exsanguinations 24 hours after treatment. The throat was opened and lungs dissected en bloc. The trachea was cannulated with polyethylene tubing and lavaged with five sequential aliquots of 3 ml NS. Lavage fluid was centrifuged at 200×g for 10 minutes. The resulting cell pellet was re-suspended in 1 ml Hank's balanced salt solution (HBSS) for performing cell count and differential. The supernatant was assayed for protein and hemoglobin as indices of acute injury. In FIG. 5 the hemoglobin content was measured in the bronchoalveolar lavage fluid 24 hours after administration of saline (control), human leukocyte elastase (HLE), HLE plus heparin, and HLE plus ODS heparin. In FIG. 6 there is shown a graph of the concentration of protein in the bronchoalveolar lavage fluid 24 hours after administration of saline (control), human leukocyte elastase (HLE), HLE plus heparin, and HLE plus ODS heparin. Lastly, in FIG. 7 there is shown a graph of the number of polymorphonuclear leukocyte (PMN) cells in the bronchoalveolar lavage fluid 24 hours after administration of saline (control), human leukocyte elastase (HLE), HLE plus heparin, and HLE plus ODS heparin. Both heparin and 2-O desulfated heparin were potent inhibitors of elastase induced injury in vivo.

The 2-O desulfated heparin from Example I has been tested for toxicity. Other sulfated polysaccharide inhibitors of elastase such as dextran sulfate, produced hemorrhage into lung air sacs (alveolar hemorrhage) when injected into rats intratracheally in doses as low as 0.5 mg/kg. The 2-O desulfated heparin from Example I produced no alveolar hemorrhage in rats even in at intratracheal doses of 10 mg/kg. The 2-O desulfated heparin from Example I can be used in humans to treat elastase mediated lung injury. As an example, for treatment of a patient with cystic fibrosis, a dose that provides a 5:1 ratio of inhibitor to protease is prepared and administered as an aerosol. In a patient producing 50 ml of sputum per day and producing average amounts of leukocyte elastase in the sputum, this dose can be about 25-250 mg of 2-O desulfated heparin administered by nebulizer over a course of 24 hours. Elastase levels in the patient's sputum can be monitored during treatment. The advantage of 2-O desulfated heparin over unmodified heparin in treating human elastase mediated lung injury is that 2-O desulfated heparin is nonanticoagulant and is less likely to cause lung hemorrhage in patients such as cystic fibrosis who are prone to coughing up blood (hemoptysis).

Materials Used in Subsequent Examples. Acetylcholine chloride, the calcium ionophore A23187, sodium nitroprusside, and indomethacin (Sigma, St. Louis, Mo.), and U-46619 (Upjohn, Kalamazoo, Mich.) were used in concentrations determined by Sato, et al. (see H. Sato, et al., Cardiovasc Res, Vol. 31, pp. 63-72 (1996). Grade I-A heparin sodium salt from porcine intestinal mucosa (Sigma) was resuspended with Krebs-Henseliet (K-H) buffer and administered as an intravenous bolus (3 mg/kg to dogs). Nonanticoagulant 2-O desulfated nonanticoagulant heparin (ODS-HEP) was synthesized according to Example I and according to Fryer, et al. (see A. Fryer, et al., supra.) from unfractionated porcine intestinal heparin 170 USP/mg anticoagulant activity and 150 U/mg anti-Xa activity. While 1.0 mg/ml of unmodified heparin inhibited 91±2% of the lysis of human red cells by canine plasma, ODS-HEP reduced erythrocyte lysis only by 4±2% at 1.0 mg/ml. ODS-HEP was resuspended in Krebs Heinseleit (K-H) buffer and administered as an intravenous bolus (3 mg/kg to dogs; 6 mg/kg to rats, with 100 μg/ml added to K-H perfusate for isolated hearts).

In Vivo Ischemia-Reperfusion Studies Performed—Surgical Procedure. All animals were handled in compliance with the Guide for the Care and Use of Laboratory Animals, published by the National Institutes of Health (NIH Publication No. 85-23, revised 1985). The Institutional Animal Care and Use Committees of Emory University and Carolinas Medical Center approved the study protocols.

Twenty-four heartworm-free adult dogs of either sex were anesthetized with sodium pentobarbital (20 mg/kg) and endotracheally intubated. Anesthesia was supplemented with fentanyl citrate (0.3 μg/kg/min) and diazepam (0.03 μg/kg/min) administered intravenously as needed to maintain deep anesthesia. Each dog was ventilated with a volume-cycled respirator using oxygen-enriched room air. A rectal temperature probe was inserted to measure core body temperature. The right femoral artery and vein were cannulated with polyethylene catheters for arterial blood sampling and for intravenous access, respectively. Serial arterial blood gases were measured to maintain the arterial oxygen tension greater than 100 mmHg. Arterial carbon dioxide tension was maintained between 30 and 40 mmHg, and arterial pH was maintained between 7.35 and 7.45 by adjustment of the ventilatory rate, and acidemia was counteracted with intravenous sodium bicarbonate.

After median sternotomy, the superior and inferior vena cava were looped with umbilical tapes and the heart suspended using a pericardial cradle. Millar catheter-tipped pressure transducers (Millar Instruments, Houston, Tex.) were placed in the proximal aorta and in the left ventricular cavity to measure aortic and left ventricular pressure, respectively. A polyethylene catheter was inserted into the left atrium for colored microsphere injection. A one centimeter portion of the left anterior descending (LAD) coronary artery distal to the first diagonal branch was dissected and loosely encircled with a 2-O silk suture. A pair of opposing ultrasonic crystals were placed intramyocardially within the proposed ischemic area at risk within the left anterior descending coronary artery distribution, and were used to assess regional function within the area at risk (see J. E. Jordan, et al., J Pharmacol Exp Therap, Vol. 280, pp. 301-309 (1997).

Experimental Protocol. Dogs were randomized to one of three groups (n=8 in each group): 1) Control (saline), 2) unmodified heparin (HEP, 3 mg/kg) and 3) modified heparin (ODS-HEP, 3 mg/kg). The LAD was occluded for 90 minutes producing ischemia and then released for four hours of reperfusion. Each pharmaceutical agent (saline, HEP, ODS-HEP) was infused as an intravenous bolus 10 minutes prior to initiation of reperfusion and at 90 and 180 minutes during reperfusion. Analog hemodynamic and cardiodyamic data were sampled by a personal computer using an analog-to-digital converter (Data Translation, Marlboro, MA). Hemodynamic and cardiodynamic data were averaged from no fewer than 10 cardiac cycles. Percent systolic shortening, segmental work, and the characteristics of segmental stiffness described by exponential curve-fitting analysis were determined as described previously in J. E. Jordan, et al., supra. Activated clotting time (ACT, in seconds) was measured throughout the experiment using the Hemochron 401 Whole Blood Coagulation System (International Technidyne, Edison, NJ). Arterial blood creatine kinase activity was analyzed using a kit from Sigma Diagnostics and expressed as international units per gram of protein. The experiment was terminated with a bolus of intravenous sodium pentobarbital (100 mg/kg). The heart was immediately excised for further analysis and placed into ice-cold Krebs-Henseleit (K-H) buffer of the following composition (mmol/L): 118 NaCl, 4.7 KCl, +1.2 KH₂PO₄, 1.2 MgSO₄.7H₂O, 2.5 CaCl₂.2H₂O, 12.5 NaHCO₃, and 11 glucose at pH 7.4.

Determination of Area at Risk Infarct Size and Regional Myocardial Blood Flow. After post-experimental excision of the heart, the myocardial area at risk and infarct size were determined as previously described (J. E. Jordan, et al., supra.) using Unisperse pigment exclusion and 1% triphenyltetrazolium chloride, respectively. The area at risk (AAR) and infarct size were calculated gravimetrically as previously described (J. E. Jordan, et al., supra.). Regional myocardial blood flow in the ischemic-reperfused and non-ischemic myocardium were obtained by spectrophotometric analyses of dye-release colored microspheres (Triton Technology, San Diego, Calif.). Left atrial injections of microspheres and reference blood sampling were performed at baseline, at the end of 90 minutes of ischemia, and at 15 minutes and four hours of reperfusion.

Measurement of Myocardial Neutrophil Accumulation. Tissue samples of 0.4 grams were taken from the non-ischemic zone and from the non-necrotic and necrotic regions of the area at risk for spectrophotometric analysis of myeloperoxidase (MPO) activity (A absorbance/minute), for assessment of neutrophil (PMN) accumulation in myocardium, as described previously (J. E. Jordan, et al., supra.).

PMN Adherence to Post-Experimental Coronary Artery Endothelium. PMN adherence to post-experimental coronary arteries was used as a bioassay of basal endothelial function. Canine PMNs were isolated from arterial blood and fluorescent labeled as previously described (see Z-Q Zhao, et al., Am J Physiol Heart Circ Physiol, Vol. 271, pp H1456-H1464 (1996). After excision of the heart, ischemic-reperfused LAD and non-ischemic left circumflex (LCx) segments were isolated, cut into 3-mm segments, opened to expose the endothelium while being submerged in ice-cold K-H buffer, and then placed in dishes containing K-H buffer at 37° C. After unstimulated, fluorescent-labeled PMNs (6×10₆ cells/dish) were incubated with post-experimental segments for 15 minutes, the coronary segments were washed of non-adherent PMNs, mounted on glass slides, and adherent PMNs were counted under epifluorescence microscopy (490-nm excitation, 504-nm emission), as described previously (see V. H. Thourani, et al., supra.).

Agonist-Stimulated Macrovascular Relaxation. Agonist-stimulated vasoreactivity in epicardial macrovessels from ischemic (LAD) and nonischemic (Lcx) was studied using the organ chamber technique (see Zhao, Z-Q, et al., Adenosine A2-receptor activation inhibits neutrophil-mediated injury to coronary endothelium. Am J Physiol Heart Circ Physiol 271:H1456-H1464, 1996). Indomethacin (10 μmol/L) was used to inhibit prostaglandin release. Coronary rings were precontracted with the thromboxane A2 mimetic U-46619 (5 nmol/L). Endothelial function was assessed by comparing the vasorelaxation responses to incremental concentrations of acetylcholine (1-686 μmol/L) and A23187 (1-191 μmol/L), whereas smooth muscle function was assessed with sodium nitroprusside (1-381 μmol/L).

In Vitro Ischemia-Reperfusion Studies Performed—PMN Degranulation. Supernatant MPO activity was measured as the product of canine PMN degranulation using the method by Ely as modified by J. E. Jordan, et al., supra.). Canine PMNs (20×10₆ cells/ml) were incubated in the presence or absence of ODS-HEP and stimulated to degranulate with platelet activating factor (PAF, 10 μmol/L) and cytochalasin B (5 μg/ml). MPO activity in supernatants was assayed spectophotometrically.

PMN Adherence to Normal Coronary Artery Endothelium. Adherence of PMNs to normal canine epicardial arteries was assessed using coronary segments and PMNs from normal animals. Unstimulated PMNs and coronary artery segments prepared and labeled as described for adherence studies were coincubated in the presence or absence of HEP or ODS-HEP. After PAF (100 nmol/L) stimulation for 15 minutes, adherent PMNs were counted as outlined earlier.

Experiments with Human Umbilical Vein Endothelial Cells (HUVEC). Primary HUVECs were isolated according to the method of Jaffe, et al., J Clin Invest Vol. 52, pp. 2745-2750 (1973), cultured on coverslips using endothelial cell growth medium (Clonetics) and tested for expression of von Willebrand's factor. HUVECs were washed twice with PBS and incubated in Neuman/Tytell medium alone for 24 hours, followed by incubation with lipopolysaccharide (1 μg/ml) plus 10-20 ng/ml TNFa for 2 hours, or in heparin or ODS-HEP (200 μg/ml) for 4 hours with the addition of lipopolysaccharide and TNFa after 2 hours. HUVECs were fixed for 20 minutes on ice with 4% paraformaldehyde in CEB (10 mmol/L Tris-HCl, pH 7.9, 60 mmol/L KCl, 1 mmol/L EDTA, 1 mmol/L dithiothreitol) with protease inhibitors, PI (1 mmol/L Pefabloc, 50 μg/ml antipain, 1 μg/ml leupeptin, 1 μg/ml pepstatin, 40 μg/ml bestatin, 3 μg/ml E-64, and 100 μg/ml chymostatin), permeabilized for 2 minutes with 0.1% NP40 in CEB/PI, washed once with cold CEB and fixed as before for 10 minutes. Coverslips were incubated in 3% H₂O₂ for 30 minutes to suppress peroxidase, washed three times in cold PBS, blocked for 2 hours with 2% bovine serum albumin (BSA) in PBS on ice and incubated overnight at 4° C. with 1 μg/ml of anti-p65 antibody (Santa Cruz Biotechnology, Santa Cruz, CA) diluted in 0.1% BSA/PBS. Unbound anti-p65 was washed away with 2% BSA/PBS and bound antibody was incubated with biotinylated swine anti-rabbit immunoglobulin (1:1000) in 0.1% BSA/PBS for 45 minutes on ice, followed by 3 washes with 2% BSA/PBS. Coverslips were then incubated with streptavidin biotin peroxidase at room temperature for 1 hour, washed again, incubated in 0.03% wt/vol 3-3′diaminobenzidine with 0.003% H₂O₂ until a brown reaction product could be seen, counterstained with eosin and viewed under light microscopy.

Electrophoretic mobility shift assays (EMSAs) were also used to study the translocation of NF-κB from the cytoplasm to the nucleus. Nuclear proteins were obtained from HUVEC as described by Digman, et al., Nucleic Acid Res Vol. 11, pp. 1475-1481 (1983) with the addition of the following proteinase inhibitors: 1 mmol/L phenylmethylsulfonyl fluoride, 1 μg/ml pepstatin A, 0.5 μg/ml chymostain, 1 μg/ml antipain, 1 μg/ml leupeptin and 4 μg/ml aprotinin. The double stranded oligonucleotide DNA probe (Santa Cruz) of the NF-κB consensus sequence AGTTGAGGGGACTTTCCCAGGC [SEQ ID NO 1] was 5′OH end-labeled with [γ³²P] ATP using polynucleotide kinase. Free radionucleotide was removed using a Sephadex G-25 column. The probe (0.5 ng) was incubated with 10 μg HUVEC nuclear protein (Bio-Rad method) in 20 μl buffer containing a final concentration of 10 mmol/L HEPES, pH 7.5, 50 mmol/L KCl, 5 mmol/L MgCl2, 1 mmol/L dithiothreitol, 1 mmol/L EDTA and 5% glycerol, plus 5 μg of poly (dI-dC) to reduce nonspecific binding. Incubations were carried out at room temperature for 20 minutes. Reactions were electrophoresed at 14 V/cm for 1.5-2.0 hours on a 6% nondenaturing polyacrylamide gel in 0.5×TBE (45 mmol/L Tris borate, 25 mmol/L boric acid, 1 mmol/L EDTA) at 4° C., and autoradiographed at −80° C.

Experiments with Isolated Perfused Rat Hearts. Male Sprague-Dawley rats (300-400 g) were anesthetized with sodium pentobarbital (40 mg/kg, i.p.), and the hearts were quickly excised and perfused in a Langendorff apparatus as previously described (Watts J A, et al., J Mol Cell Cardiol, Vol. 31, pp. 1653-1662 (1999) with modified Krebs-Henseleit bicarbonate buffer (KHB), consisting of (in mmol/L): 118 NaCl, 4.7 KCl, 1.2 KH₂PO₄, 1.2 MgSO₄.7H₂O, 3.0 CaCl₂.2H₂O (yielding 2.5 mmol/L free Ca²⁺ in the presence of EDTA), 0.5 EDTA, 11 dextrose, and 25 NaCHO₃. Three groups were studied: 1) nonischemic control hearts were perfused 45 minutes; 2) ischemic-reperfused hearts were subjected to 15 minutes warm global ischemia and 15 minutes reperfusion; and 3) ODS-HEP hearts from rats injected with 6 mg/mg ODS-HEP i.v. 120 minutes before heart excision were subjected to 15 minutes each of global ischemia and reperfusion, with 100 μg/ml ODS-HEP in perfusion buffer. After perfusion, ventricles were frozen with Wollenberger clamps precooled in liquid N₂, and pulverized under liquid N₂. Nuclear proteins were immediately isolated from frozen myocardial powders by the method of Li, et al., Am JPhysiol Heart Circ Physiol, Vol. 276, pp. H543-H552 (1999). EMSAs were performed using 15 μg of nuclear protein (Pierce protein assay) in each binding reaction. Competition experiments were performed by incubation of nuclear proteins with 10× unlabeled NF-κB or cyclic-AMP responsive element oligonucleotides (CRE, AGAGATTGCCTGACGTCAGAGAGCTAG) [SEQ ID NO 2] for 5 minutes prior to addition of ³²P-labeled NF-κB probe. Supershift assays were performed by adding 0.5 μg of antibodies to p65 and p50 components of NF-κB (Santa Cruz) to the binding reaction after labeled probe. Reactions were electrophoresed at 100 V for 2 hours at room temperature on a 5% nondenaturing polyacrylamide gel in 0.5×TGE (120 mmol/L glycine, 1 mmol/L EDTA, 25 mmol/L Tris, pH 8.5) and autoradiographed.

The data were analyzed by one-way analysis of variance or repeated measures two-way analysis of variance for analysis of group, time and group-time interactions. If significant interactions were found, Tukey's or Student-Newman-Keuls post hoc multiple comparisons tests were applied to locate the sources of differences. Differences in the densities of the p65-containing NF-κB gel band between treated and untreated ischemic reperfused rat hearts were compared using the t test. A p<0.05 was considered significant, and values are expressed as mean±standard error of the mean (SEM).

### EXAMPLE IV

2-O Desulfated Heparin Reduces Infarct Size. Using the procedures described above, heparin and 2-O desulfated heparin significantly reduced myocardial infarct size. As shown in FIG. 8, the area at risk (AAR) is expressed as a percentage of the left ventricle (LV) at risk for infarction. The infarct size (area of necrosis, AN) is expressed as a percentage of the area at risk (AAR). *p<0.05 versus Control. Heparin (HEP) or 2-0 desulfated heparin (ODS-HEP) treatment decreased infarct size (area of necrosis, AN), expressed as a percentage of the area at risk (AN/AAR), by 35% and 38%, respectively, compared to Controls. There was no statistical difference in size of infarcts between the HEP and ODS-HEP groups, and the area at risk from LAD occlusion, expressed as a percentage of the left ventricular mass (AAR/LV), was comparable among groups.

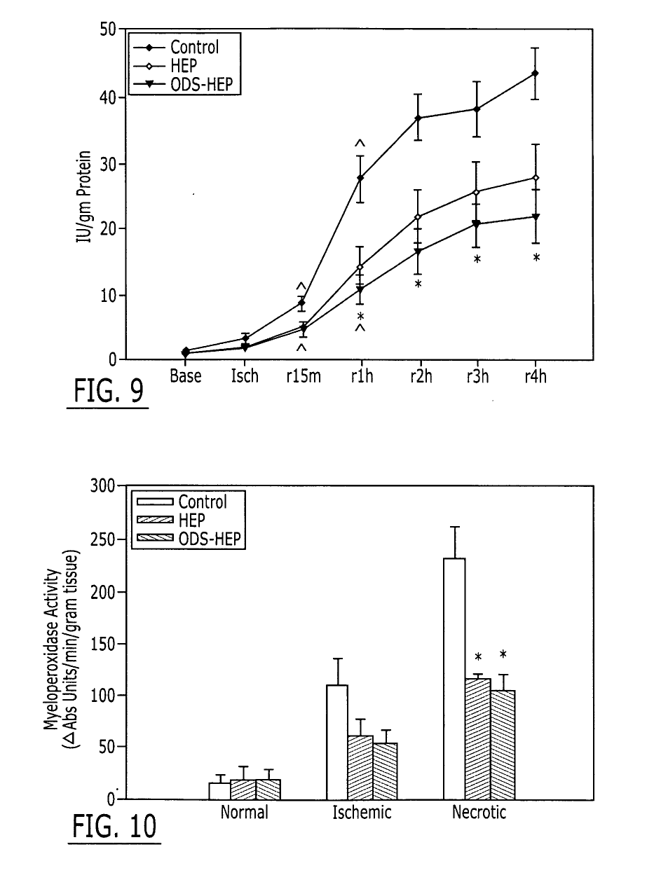

As shown in FIG. 9, plasma creatine kinase (CK) activity was used to confirm histologic measurement of infarct size during the time course of the experiment. *p<0.05 HEP and OPS-HEP versus Control. There were no significant differences in plasma CK activity at baseline among groups and no increases in CK activity after regional ischemia. Hearts in the Control group showed a steep rise in CK activity within the initial hour of reperfusion, which was significantly reduced by HEP or ODS-HEP treatment, consistent with the smaller infarct sizes in these groups (CK after 4 hour reperfusion=43.4±3.7 for Control; 27.6±5.3 for HEP; and 21.9±4.0 international units/g protein for ODS-HEP).

Despite their favorable effects on infarct size, HEP and ODS-HEP produced no significant changes in myocardial blood flow. Subendocardial blood flow in the ischemic-reperfused LAD coronary artery region was statistically comparable among the three groups at baseline. Regional myocardial myocardial blood flow in the distribution of the non-ischemic-reperfused left circumflex (LCx) coronary artery. Transmural blood flow in the area at risk was significantly decreased during ischemia, with no group differences. All groups showed a comparable hyperemic response in the area at risk at 15 minutes of reperfusion, after which blood flow was diminished to similar levels in all groups by four hours. In the non-ischemic-reperfused LCx coronary artery region, transmural blood flow was comparable in all groups throughout the protocol.

Differences in infarct size were also not from hemodynamic or cardiodynamic differences. Hemodynamics at baseline and during ischemia and reperfusion were comparable among groups (data not shown). Heart rate was significantly increased during ischemia and reperfusion in all animals, and left ventricular end diastolic pressure was comparably elevated during ischemia in all three groups. Following ischemia, hearts in all groups demonstrated dyskinesis in the area at risk. All hearts showed poor recovery of percent systolic shortening throughout the four hours of reperfusion (−6±2% for Control hearts; −7±3% for HEP treated hearts; and −6±4% for ODS-HEP treated hearts at 4 hour reperfusion), and diastolic stiffness (as measured by the valueless β-coefficient) increased following ischemia to comparable levels in all groups (from 0.2±0.05 at baseline to 0.7±0.1 units after 4 hour reperfusion in Control hearts; from 0.2±0.04 at baseline to 1.0±0.2 units after 4 hour reperfusion in HEP treated hearts; from 0.2±0.04 at baseline to 0.5±0.2 units after 4 hour reperfusion in ODS-HEP treated hearts).

### EXAMPLE V

Heparin and 2-O Desulfated Heparin Reduce PMN Accumulation in Reperfused Myocardium. Using the procedures described above, heparin and 2-O desulfated heparin were found to reduce PMN accumulation in reperfused myocardium. PMN influx is a major mechanism underlying lethal reperfusion injury. Treatment with HEP or ODS-HEP significantly reduced myeloperoxidase (MPO) activity in necrotic myocardium by 50% compared to the Control group as shown in FIG. 10. In FIG. 10 myeloperoxiase activity, an index of PMN accumulation, is shown in normal ischemic, and necrotic myocardial tissue samples from each group. *p<0.05 HEP and ODS-HEP versus Control. PMN accumulation within normal myocardium was low and comparable among Control, HEP and ODS-HEP groups (16±8, 18±11, and 18±86 absorbance units/minute, respectively). HEP and ODS-HEP both decreased MPO activity in the non-necrotic area at risk, but these changes did not achieve significance (p>0.10).

### EXAMPLE VI

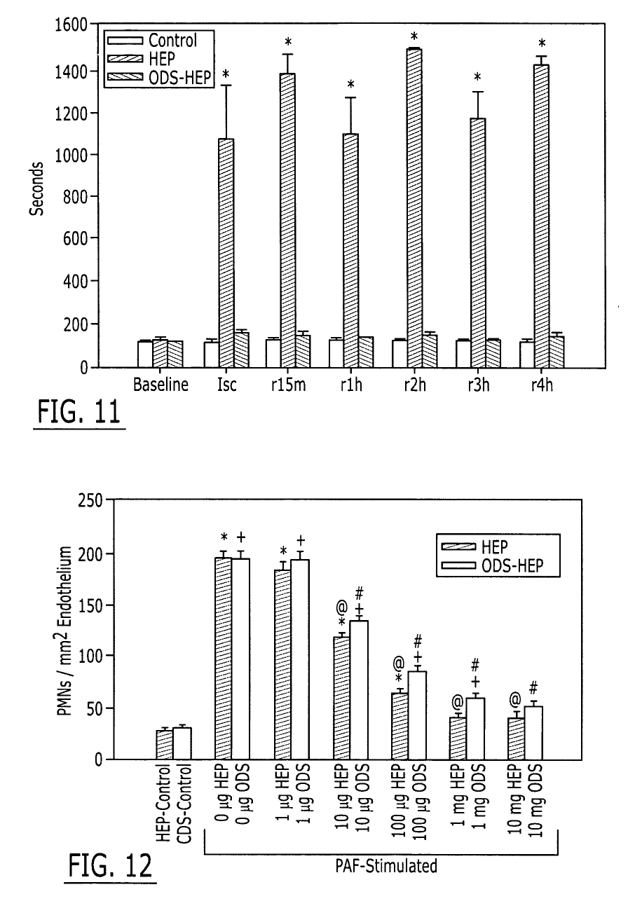

2-O Desulfated Heparin Does Not Produce Anticoagulation. Despite reducing infarct size, ODS-HEP did not produce anticoagulation. As shown in FIG. 11, systemic whole blood anticoagulation was studied using the activated clotting time, measured in seconds. *p<0.05 HEP versus other groups. At four hours of reperfusion, activated clotting time (ACT) was increased greater than ten-fold after HEP treatment compared with Control (1425±38 seconds versus 123±10 seconds, respectively). In contrast, ACT in the ODS-HEP group (145±10 seconds) was not different from Controls (123±10 seconds, p=0.768). Thus, ODS-HEP was able to affect the same benefits as HEP without anticoagulation.

### EXAMPLE VII

Heparin and 2-O Desulfated Heparin Reduce Neutrophil Adherence and Endothelial Dysfunction in Coronary Arteries. This example shows that heparin and 2-O, 3-O desulfated heparin reduce neutrophil and endothelial dysfunction in coronary arteries. ODS-HEP did not significantly reduce PAF-stimulated PMN degranulation, suggesting that ODS-HEP has little direct effect on PMN activity. However, PAF-stimulated PMN attachment to coronary endothelium was significantly reduced by both HEP and ODS-HEP in a dose-dependent manner (FIG. 12). Neutrophil adherence to normal coronary endothelium was stimulated by 100 nM platelet activating factor (PAF) added to medium and was inhibited in a dose-dependent manner by HEP or ODS-HEP. *p<0.05 HEP group versus HEP control, @p<0.05 HEP group versus 0 mg HEP group, +p<0.05 ODS-HEP versus ODS control and #p<0.05 ODS-HEP versus 0 mg ODS group. Inhibition of PMN adherence to PAF-stimulated coronary endothelium was charge dependent, as suggested by reversal of the inhibiting effects of the polyanions HEP or ODS-HEP on attachment by the polycation protamine (PMNS/mm² endothelium=66±3 with 100 μg/ml HEP versus 180±8 with HEP+1 mg/ml protamine; 86±4 with 100 μg/ml ODS-HEP vs 136±4 with ODS-HEP+1 mg/ml protamine; p<0.05 for both).

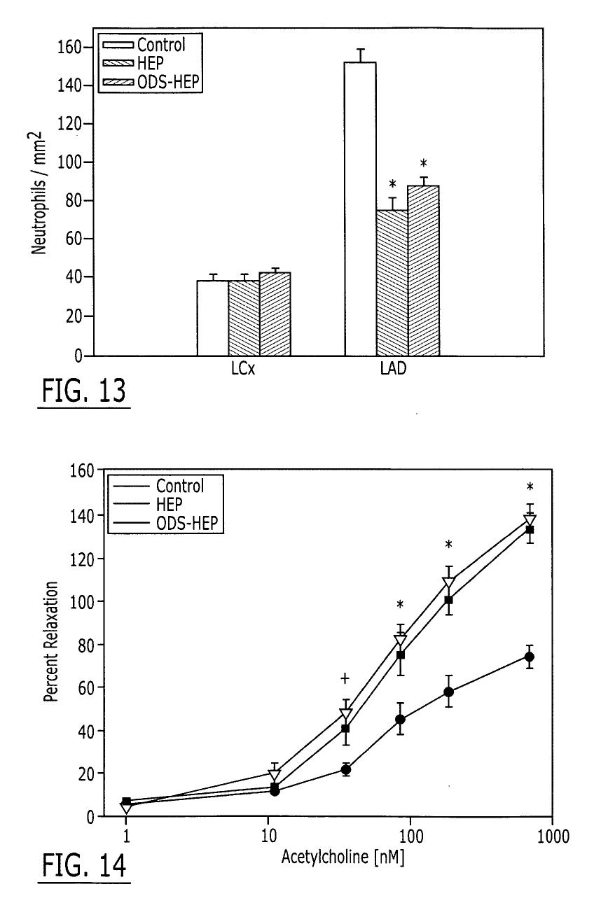

HEP and ODS-HEP also reduced PMN adherence to ischemic-reperfused coronary endothelium in vivo. The bar graph in FIG. 13 shows that PMN adherence to the ischemic-reperfused LAD coronary artery was increased by 300% in the untreated Control group compared to the non-ischemic-reperfused LCx artery. Neutrophil (PMN) adherence to the coronary endothelium was quantitated as the number of adherent PMNs/mm² of coronary endothelium. LCx=the non-ischemic-reperfused left circumflex coronary artery, LAD=the ischemic-reperfused left anterior descending coronary artery. *p<0.05 HEP and ODS-HEP versus LAD control. HEP or ODS-HEP reduced PMN adherence to the ischemic-reperfused LAD by 51 and 42%, respectively, compared to untreated Controls (FIG. 13).

HEP and ODS-HEP also preserved receptor-mediated vasodilator responses of coronary endothelium following ischemia and reperfusion. To quantify agonist-stimulated endothelial dysfunction in epicardial coronary arteries, the vascular response to incremental concentrations of the vasodilators acetylcholine (endothelial-dependent; receptor-dependent), A23187 (endothelial-dependent; receptor-independent), and sodium nitroprusside (direct smooth muscle) in post-ischemic coronary vascular ring preparations was studied.

FIG. 14 illustrates vasodilator responses to acetylcholine in isolated coronary rings from the ischemic-reperfused LAD, expressed as a percentage of U46619-induced precontraction. In the Control group, there is a statistically significant shift to the right in the concentration-response curve, representing reduced relaxation to acetylcholine. In contrast, the relaxant effect of coronary vessels to acetylcholine was preserved by HEP or ODS-HEP-treatment. Response curves are shown to incremental concentrations of acetylcholine (Ach) in the ischemic-reperfused left anterior descending (LAD) coronary artery precontracted with U46619. *p<0.05 HEP and ODS-HEP versus Control and *p <0.05 HEP versus Control.

The concentration of acetylcholine required to effect 50% relaxation (EC₅₀ log [M]) was significantly greater for the Control (−6.98±0.06) compared to the HEP (−7.30±0.06) or ODS-HEP (−7.20±0.05) groups (p<0.05). There were no differences in non-ischemic-reperfused ring preparations from LCx. In addition, there were no differences between LAD versus LCx vasodilator responses to incremental concentrations of A23187 (maximal relaxation=122±4 and 120±7% and EC₅₀ log [M]=−7.18±0.06 and −7.17±0.09 for LAD and LCx, respectively) or sodium nitroprusside (maximal relaxation=129±5 and 121±4% and EC₅₀ log [M]=−7.31±0.02 and −7.29±0.04 for LAD and LCx, respectively), and responses were unaffected by HEP or ODS-HEP.

### EXAMPLE VIII

2-O Desulfated Heparin Prevents Activation of Nuclear Factor-KB. This example shows that 2-O desulfated nonanticoagulant heparin prevents activation of nuclear factor-κB. This transcription factor, which regulates expression of a host of pro-inflammatory cytokines, is resident in the cytoplasm in unstimulated cells, but migrates to the nucleus when activated, there binding to its regulatory consensus sequence and fostering cytokine expression. NF-κB is held in the cytoplasmic compartment of cells by its inhibitor, I-κB, to which it is physically attached. NF-κB is cytosolic when complexed with its inhibitor, IκB, but is activated by phosphorylation, ubiquitination and proteolytic degration of IκB. Release from IκB exposes the NF-κB nuclear localization sequence (NLF), a highly cationic domain of eight amino acids (VQRDRQKLM, single-letter amino acid code) that targets nuclear translocation. NF-κB is activated in the heart by ischemia or ischemia and reperfusion (see C. Li, et al., supra). Nuclear translocation of NF-κB is prevented by synthetic cell permeable peptides containing the NF-κB NLF, which competes for nuclear uptake (see Y-Z, Lin, et al., J Biol Chem, Vol. 270, pp. 14255-14258 (1995). Heparin is readily bound and internalized into the cytosolic compartment by endothelium, vascular and airway smooth muscle, mesangial cells and even cardiac myocytes. Once internalized into the cytoplasm it was postulated that the polyanion heparin might bind electrostatically to the positively charged amino acids of the NLF and prevent it from targeting NF-κB to the nuclear pore.

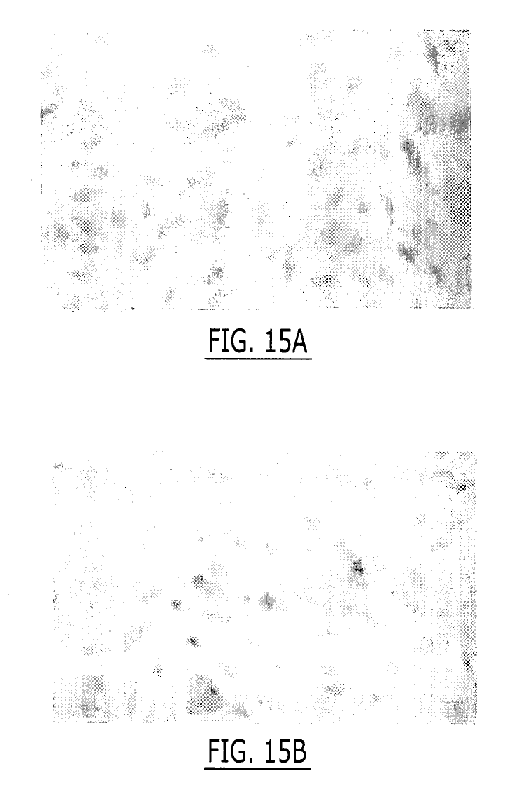

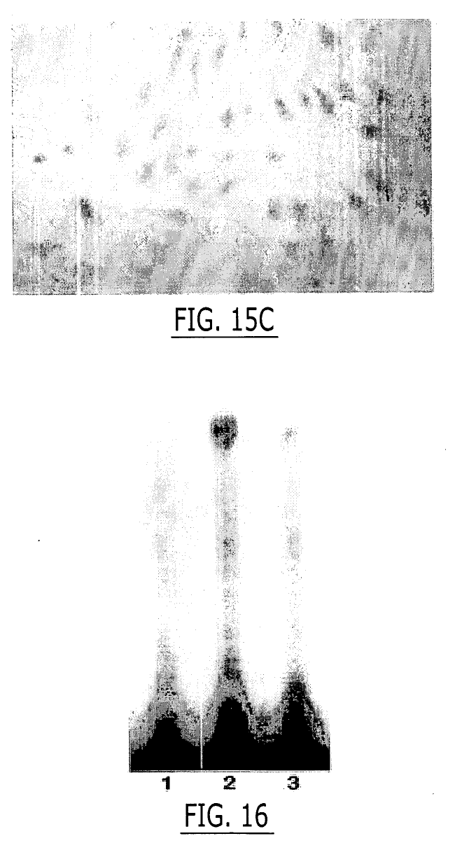

The increase in PMN adherence following ischemia-reperfusion is from enhanced expression of endothelial cell adhesion molecules, the transcription of which are strongly influenced by activation of the nuclear transcription factor NF-κB as a consequence of myocardial ischemia-reperfusion (see C. Li, et al., supra). To study whether heparin could inhibit activation of NF-κB, immunohistochemical staining for NF-κB in human umbilical vein endothelial cells (HUVECs), with and without stimulation or pretreatment with ODS-HEP were performed. FIG. 15A shows that in the unstimulated state, nuclear factor-KB is normally present only in the cytoplasm of HUVECs, but not in nuclei. In HUVECs stimulated with tumor necrosis factor α (TNFα) without addition of heparin, nuclei stain positive (brown) for the p65 component of NF-κB (FIG. 15B), corresponding to translocation of NF-κB from the cytoplasm to the nucleus. However, in HUVECs pre-treated with 200 μg/mL 2-O desulfated heparin, TNFα stimulation fails to produce translocation of NF-κB from cytoplasm to the nucleus (FIG. 15C).

Interruption of endothelial NF-κB activation by heparin and 2-O desulfated heparin was confirmed by electrophoretic mobility shift assays (EMSAs) as shown in FIG. 16. Tumor necrosis factor (TNF) stimulates endothelial DNA binding of NF-κB (FIG. 16, lane 2) compared to untreated controls (lane 1). Pretreatment with 200 μg/ml ODS-HEP eliminates NF-κB binding activity (lane 3), indicating that ODS-HEP prevents activation of NF-κB. HUVECs were stimulated with 10 ng/ml TNFα for one hour and nuclear protein was harvested for electrophoretic mobility shift assays to detect binding of NF-κB, using the oligonucleotide consensus AFTTGAGGGGACTTTCCCAGGC [SEQ ID NO. 1], end-labeled with [γ³²P] ATP. Treatment of monolayers with TNF stimulates DNA binding of NF-κB (lane 2) compared to untreated controls (lane 1). Pretreatment of cells with 200 μg/ml ODS-HEP virtually eliminates NF-κB binding activity in nuclear protein extracts (lane 3), confirming that 2-O desulfated heparin prevents translocation of NF-κB from the cytoplasm to the nucleus.

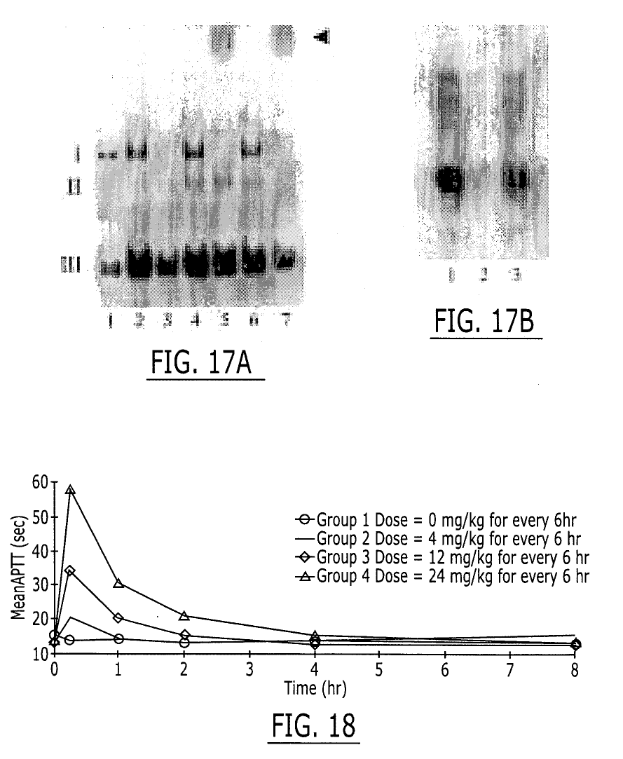

2-O desulfated nonanticoagulant heparin also reduced DNA binding of NF-κB in ischemic-reperfused myocardium. Exposure of rat hearts to 15 minute warm global ischemia and 15 minute reperfusion increased DNA binding of myocardial nuclear protein to oligonucleotide sequences for NF-κB (FIG. 17A, lane 2). Three distinct bands of increased DNA binding were observed, all of which were eliminated by addition of excess unlabeled NF-κB oligonucleotide probe. Supershift experiments identified complex I as the band containing the p65 component of NF-κB (FIG. 17, lane 5). ODS-HEP treatment reduced ischemia-reperfusion related stimulation of NF-κB binding to DNA in all three bands (FIG. 17, lane 3). DNA binding of the p65-containing complex I was nearly eliminated by ODS-HEP, with a reduction of 54±6% as measured by densitometry in comparison to complex I of untreated ischemic-reperfused rat hearts (p<0.05, n=4). Thus, in addition to directly attenuating vascular adherence of PMNs to coronary endothelium, decreasing PMN accumulation in the area at risk and reducing myocardial necrosis, HEP or ODS-HEP also interrupt NF-κB activation and possibly adhesion molecule and myocardial cytokine expression.

Langendorf perfused rat hearts were subjected to 15 minutes of warm global ischemia followed by 15 minutes of reperfusion. Nuclear protein was then harvested for EMSAs to measure DNA binding of NF-κB. Compared to sham perfused control hearts (FIG. 17A, lane 1), ischemia and reperfusion typically increased DNA binding of myocardial nuclear protein to oligonucleotide sequences for NF-κB (lanes 2 and 4). Three distinct complexes were identified. Supershift experiments performed with antibody to p65 (lane 5), antibody to p50 (lane 6) or both antibodies (lane 7) demonstrated complex I to be shifted (arrow), identifying it as the band containing the p65 component of NF-κB. Pretreatment and perfusion with ODS-HEP (6 mg/kg iv 2 hours prior to heart perfusion; 100 μg/ml in perfusate) prevented the ischemia-reperfusion related stimulation of NF-κB DNA binding of the p65-containing complex I (lane 3). DNA binding of the p65-containing complex I was nearly eliminated by ODS-HEP, with a reduction of 54±6% as measured by densitometry in comparison to complex I of untreated ischemic-reperfused rat hearts (p<0.05, n=4). At right in FIG. 17B is shown a competition experiment in which nuclear proteins were incubated with 10× unlabeled NF-κB (lane 2) or cyclic AMP response element oligonucleotides (CRE, AGAGATTGCCTGACGTCAGAGAGCTAG [SEQ ID NO 2], lane 3) for 5 minutes before addition of labeled NF-κB probe. Compared with binding reactions without excess probe (lane 1), addition of unlabeled NF-κB blocked DNA binding in all three complexes.

### EXAMPLE IX

Reduction of Contractile Dysfunction Following Ischemia and Reperfusion of Isolated Rat Hearts by 2-O Desulfated Heparin. This example shows that 2-O desulfated heparin reduces contractile dysfunction following ischemia and reperfusion of isolated rat hearts. After 15 minutes of both ischemia and reperfusion, hearts recovered high contractile function (95% of baseline, ischemia-reperfusion; and 93% of baseline ODS-HEP ischemia-reperfusion). Therefore, in additional studies, the period of ischemia was increased to 30 minutes. Both untreated and ODS-HEP treated hearts had reduced contractile function after 30 minutes of ischemia and 15 minutes of reperfusion (Pressure Rate Product=36,780±2,589 for Sham versus 4,575±1,856 for Ischemic-Reperfused and 10,965±2,908 mm Hg/min for ODS-HEP treated Ischemic-Reperfused hearts, n=4 each), but hearts treated with ODS-HEP had significantly improved recovery of contractile function, which was 2.4 times better than that observed in hearts that did not receive ODS-HEP (p<0.05). Thus, in this severe model, ODS-HEP reduces both molecular and physiologic consequences of ischemia and reperfusion.

### EXAMPLE X

Dose-Response Effect of 2-O Desuflated Heparin on the Activated Partial Thromboplastin Time. This example shows the dose of 2-O desulfated heparin that must be administered to dogs to produce anticoagulant effects. The usual anticoagulating dose of unfractionated porcine intestinal heparin is 0.5 mg/kg intravenously as a bolus, followed by an infusion of about 0.1 mg/kg/hour. To determine the effect of 2-O desulfated heparin on anticoagulation in dogs, a ten-day multiple dose study of intravenous 2-O desulfated heparin was performed in adult Beagle dogs. Animals were given drug as a 50 mg/ml formulation intravenously at doses infused every 6 hours for a total of 10 days. Clinical signs, body weight, feed consumption, clinical chemistries, hematologic parameters, urine analysis, prothrombin time and activated partial thromboplastin time (APTT) were monitored. At the end of the study animals were euthanized and necropsy was performed to examine for gross organ pathology. Four dose levels were examined: 0 mg/kg every 6 hours (Control); 4 mg/kg every 6 hours; 12 mg/kg every 6 hours; and 24 mg/kg every 6 hours. Three dogs were studied at each dosing level, and animals were dosed for 10 consecutive days. Blood was sampled for measurement of APTT immediately before the last dose, and at 15 minutes, 1, 2 4, and 8 hours after the last dose of drug. APTT values were analyzed using computerized compartmental modeling using WinNonlin software, the Gauss-Newton method and PK model 2, assuming a one-compartment intravenous infusion, no lag time and 1 st order elimination, to model pharmacokinetic parameters appropriate to each dose of drug. The time points were transformed to 0 (time of dosing), 0.25, 1, 2, 4, and 8 hours. Deviation from baseline values of APTT was analyzed to minimize parameter estimate errors. Baseline (t=0) mean APTT values for groups treated with 4, 12 or 24 mg/kg were 13.48, 13.39 and 13.58 seconds, respectively.

Results are shown in FIG. 18. Progressively larger doses of 2-O desulfated heparin prolonged the APTT within the first half hour after injection to progressively longer times. Compartmental modeling values for each dose are shown below in Table I.

| | | | | | | Coefficient of |
|---|---|---|---|---|---|---|
| Dose | | Parameter | Units | Estimate | Std Error | Variation (%) |
| 4 | mg/kg | AUC | hr*sec | 2.992568 | 0.706449 | 23.61 |
| 4 | mg/kg | K10_HL | hr | 0.231056 | 0.079270 | 34.31 |
| 4 | mg/kg | Cmax | sec | 6.315781 | 0.514230 | 8.14 |
| 4 | mg/kg | CL | mg/(hr*sec)/hr | 1.336645 | 0.315854 | 23.63 |
| 4 | mg/kg | AUMC | hr*hr*sec | 1.371626 | 0.655527 | 47.79 |
| 4 | mg/kg | MRT | hr | 0.333344 | 0.114363 | 34.31 |
| 4 | mg/kg | Vss | mg/(sec)/kg | 0.445562 | 0.065059 | 14.60 |
| 4 | mg/kg | AUC | hr*sec | 17.019598 | 0.709037 | 4.17 |
| 12 | mg/kg | K10_HL | hr | 0.478107 | 0.024939 | 5.22 |
| 12 | mg/kg | Cmax | sec | 20.697613 | 0.427065 | 2.06 |
| 12 | mg/kg | CL | mg/(hr*sec)/kg | 0.705070 | 0.029403 | 4.17 |
| 12 | mg/kg | AUMC | hr*hr*sec | 13.866942 | 1.154051 | 8.32 |
| 12 | mg/kg | MRT | hr | 0.689763 | 0.035980 | 5.22 |
| 12 | mg/kg | Vss | mg/(sec)/kg | 0.486331 | 0.012126 | 2.49 |
| 24 | mg/kg | AUC | hr*sec | 41.736291 | 1.944886 | 4.66 |
| 24 | mg/kg | K10_HL | hr | 0.563503 | 0.032729 | 5.81 |
| 24 | mg/kg | Cmax | sec | 44.195335 | 1.058431 | 2.39 |
| 24 | mg/kg | CL | mg/(hr*sec)/kg | 0.575039 | 0.026823 | 4.66 |
| 24 | mg/kg | AUMC | hr*hr*sec | 39.147103 | 3.675518 | 9.39 |
| 24 | mg/kg | MRT | hr | 0.812963 | 0.047218 | 5.81 |
| 24 | mg/kg | Vss | mg/(sec)/kg | 0.467486 | 0.013177 | 2.82 |

Throughout the 10 day study, anticoagulation effect declined within less than 2 hours and the APTT had fallen back to baseline values prior to each subsequent dose, so that there was no accumulation of drug effect after any dosing interval. An examination of the data in Table I demonstrated that as the bolus size is increased, the rate of drug clearance progressively decreases. Therapeutic anticoagulation is defined clinically as prolongation of the testing parameter to 2-2½ times control value. This would suggest clinically effective anticoagulation at 27 to 34 seconds APTT in a treated dog. After a bolus dose of 12 mg/kg of 2-O desulfated heparin, the mean peak APTT at 15 minutes was 31 seconds. From the decay curve, the estimated rate of clearance of the 12 mg/kg dose was 0.71 mg/kg/hr. Therefore, effective anticoagulation can be achieved by an intravenous bolus loading infusion of about 12 mg/kg, followed by a constant intravenous infusion of about 0.7 to 0.8 mg/kg/hour, adjusted upward or downward to achieve an APTT of 2-2½ times control (or about 50-80 seconds in a human).

### EXAMPLE XI

Dose-Response Effect of 2-O Desulfated Heparin as an Anti-Inflammatory Agent. To establish the dose at which 2-O desulfated heparin exhibits significant and clinically meaningful anti-inflammatory activity, the drug was studied in a closed-chest porcine model of myocardial infarction. A closed-chest pig infarction model developed by Dr. Vinten-Johansen as a strenuous test of the ODSH dose required to block neutrophil influx from serious vascular inflammation was used. Compared to the open-chest dog, the closed-chest porcine model is less invasive and more closely simulates the clinical scenario, particularly in human patients undergoing coronary angioplasty for acute myocardial infarction. Just as in the canine model, the closed chest pig model is characterized by reproducible infarcts that can be reduced by a number of interventions, including ischemic pre-conditioning or intracoronary adenosine. There is prominent influx of neutrophils into reperfused myocardium.

Farm-bred pigs weighing ˜35 kg were initially anesthetized using a cocktail of ketamine, xylazine, acepromazine, diazepam and atropine, followed by maintenance anesthesia with inhaled isoflurane. Intravenous amiodarone was administered at 7-8 mg/kg over the entire case to control arrhythmias due to the coronary occlusion or subsequent reperfusion. The use of amiodarone has reduced the incidence of fatal arrhythmias in this model from 50% to 12% in studies we have conducted over the past eighteen months. To prevent thrombi from forming on intravascular catheters, aspirin (325 mg) and unfractionated heparin (50 U/kg intravenously) was administered after induction of anesthesia to provide a level anti-coagulation similar to that received currently by patients experiencing myocardial infarction and undergoing emergency balloon angioplasty to dislodge coronary occlusion. The bolus of heparin was repeated every 90 minutes, the average half-life of unfractionated heparin. Electrocardiographic electrodes were placed subdermally for limb lead II EKG. Through a left femoral artery cut-down, a pig-tail catheter with high-fidelity solid state transducers in the ventricular and arterial positions were fluoroscopically guided into the left ventricle for injection of microspheres. A similar cut-down was performed in the contralateral femoral artery into which is placed a sheath by which to introduce a 7-Fr guide catheter and angioplasty-type balloon catheter. The 7-Fr guide catheter was inserted through this sheath and fluoroscopically guided to the left main coronary artery (LAD). The LAD was angiogramed and sized for appropriate balloon catheter to ensure complete occlusion of the vessel during inflation. The left main coronary ostium was engaged by the catheter, and an angioplasty-type balloon catheter was guided into the LAD just distal to the first diagonal branch using a guide wire. Placement of the balloon was verified by intracoronary contrast dye injection, and documented by film capture. After placement of the intracoronary catheter, the animal was allowed to stabilize for 10 minutes. A baseline left ventriculogram was performed in the catheterization laboratory. Then hemodynamics (left ventricular and arterial pressures, heart rate) were measured at baseline. Global as well as segmental wall motion was determined, the latter in anterior, antero-lateral and antero-septal aspects, compared to posterior segments. In addition, echocardiograms were taken for later assessment of global and regional wall motion. Microspheres were injected at baseline via the pig-tail catheter to quantify baseline myocardial blood flow during steady-state, while a reference sample was withdrawn simultaneously from the femoral artery through the side port of the sheath. The angioplasty balloon was inflated to totally occlude the mid-LAD coronary artery (distal to the 1st or 2nd diagonal branch, depending upon anatomical considerations), and occlusion was maintained for 75 minutes, targeting an infarct size of approximately 50% of the area at risk. If ventricular fibrillation occurred, DC countershocks were delivered by external paddles to convert the heart to normal sinus rhythm. Inflation and position of the balloon were verified by contrast angiogram. Microspheres were injected at the end of the ischemic period to quantify collateral blood flow to the area at risk, which is used as a covariate to infarct size.

After 75 minutes of balloon inflation, the pigs were randomly assigned to one of four groups:

| Group | Treatment | # Experiments |
|---|---|---|
| 1 | Vehicle Control | 6 |
| 2 | 5.0 mg/kg 2-O desulfated heparin | 6 |
| 3 | 15.0 mg/kg 2-O desulfated heparin | 6 |
| 4 | 45.0 mg/kg 2-O desulfated heparin | 6 |

Saline vehicle or 2-O desulfated heparin were administered intravenously 2 minutes before deflation of the intracoronary balloon and re-administered every 90 minutes after the onset of reperfusion. The 2-O desulfated bulk drug has been produced under GMP conditions in a 1.3 kg lot by Scientific Protein Laboratories. The formulation used was 2-O desulfated heparin at a concentration of 50 mg/ml in USP sterile water with addition of 0.4% NaCl to adjust to about 280-300 mOsm per ml and NaOH to adjust to pH of 6.0 to 7.0. This formulation has been prepared by BioConcept Laboratories, Inc., Derry, NH, has officially passed release testing, and is stable in accelerated testing.

After microsphere injection and sampling, the intracoronary balloon was deflated to initiate reperfusion, and reperfusion was continued for a total of 3 hours. EKG tracings (and all hemodynamic and cardiodynamic data) were acquired 1 minute before and 1 minute after each administration of the drug/vehicle. Hemodynamic, cardiodynamic and EKG data were acquired, blood samples were drawn for measurement of creatine kinase and activated clotting time (ACT), and microspheres were administered/sampled at 15, 60, 120 and 180 minutes of reperfusion. A repeat ventriculogram was captured at the end of 180 minutes of reperfusion. The animal was euthanized and the heart excised for processing.

End-Point Measurements.
1) The major anti-inflammatory end-point was Infarct size, assessed as the ratio of area of necrosis (AN) to area at risk (AAR);
2) Activated clotting times at baseline, end ischemia, 15, 60, 120, 180 and 240 minutes of reperfusion;
3) Hemodynamic variables including heart rate, left ventricular pressure and arterial pressure;
4) Electrocardiographic data taken at baseline, before and after each administration of drug (2 minutes prior to balloon deflation, each 90 minutes thereafter), and at each hour of the 4 hours of reperfusion;
5) Incidence of ventricular fibrillation, the number and voltage parameters of counter shocks, and any additional dosages of amiodarone and/or lidocaine;
6) Cardiodynamic function including maximal rate of increase in left ventricular pressure, anterior (LAD) segmental wall motion and global ejection fraction by ventriculogram (baseline and 180 minutes of reperfusion); and
7) Myocardial blood flow by microspheres (collateral blood flow in area at risk)

Area at risk and infarct size. The area at risk was identified using intracoronary injection of Unisperse blue dye. The angioplasty balloon catheter was re-inflated in its original position, and the left main and right coronary artery ostia were sequentially engaged by guide catheters. 15 ml of Unisperse Blue dye was injected into each vascular tree to stain the normally perfused region blue and thereby demarcate the area at risk (AAR). The heart was then rapidly excised after euthanasia, and the left ventricle (LV) was cut into 4-5 mm thick transverse slices. The AAR was separated from the non-ischemic zone and incubated for 15 minutes in 1% solution of triphenyltetrazolium chloride (Sigma Chemical, St. Louis, Mo.) at 37° C. to differentiate the necrotic zone (pale) from the ischemic non-necrotic zone (red). The AAR was calculated as the sum of the weights of the non-necrotic and necrotic tissue within the ischemic zone, divided by the weight of the LV and expressed as a percentage (AAR/LV). The infarct size was calculated as the weight of necrotic tissue divided by the weight of the area at risk (AN/AAR) and expressed as a percentage.

Statistical Analysis. Analysis of variance (ANOVA) for repeated measures was be used to analyze group-time interactions in hemodynamics and regional wall motion. Post hoc analysis when significant overall differences are found was performed by Student Neuman Keul's multiple comparisons test. Discrete endpoints were analyzed using one-way ANOVA and post hoc Student Neuman Keul's multiple comparisons test. A p-value of <0.05 was accepted as statistically significant after testing for normality of data.

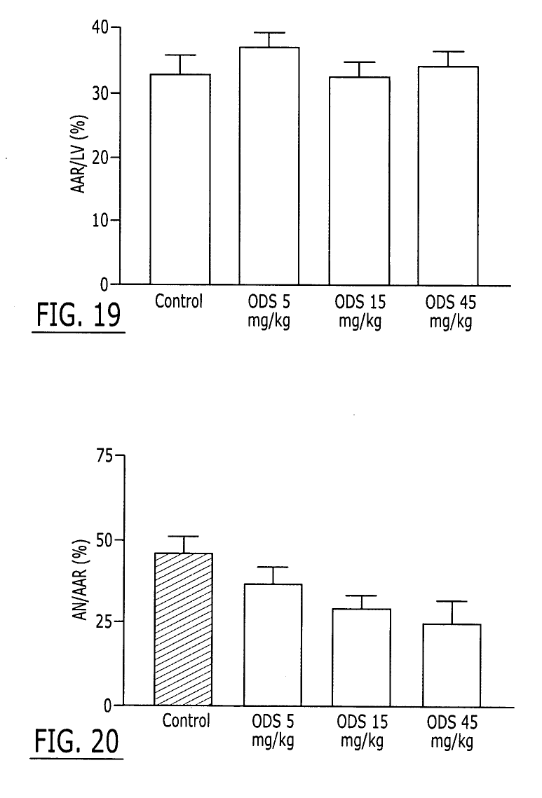

FIG. 19 shows that there was no significant difference among experimental groups in the area at risk (AAR) as a percentage of left ventricular mass. Infarctions of approximately 40% of the left ventricular mass were consistently produced by this model in control animals. In contrast, as shown in FIG. 20, infarct size defined as a percentage of the area at risk was reduced in a dose-dependent fashion by treatment of pigs with 5, 15 or 45 mg/kg 2-O desulfated heparin immediately before reperfusion, and again after 90 minutes.

Analysis of the dose-response relationships for anticoagulant effect in Example X andanti-inflammatory effect in Example XI demonstrate a convergence at about 12-15 mg/kg. Therefore, to achieve an optimum level of anticoagulation, accompanied by a greatly enhanced anti-inflammatory effect compared to that demonstrated by anticoagulation with unfractionated heparin, a human might be treated with a loading intravenous bolus dose of 12-15 mg/kg of 2-O desulfated heparin, administered without admixture with unfractionated heparin, followed immediately by initiation of an infusion of 2-O desulfated heparin at a rate of 0.7 to 3.0 mg/kg/hour, adjusted upward or downward to achieve an APTT of 2 to 2½ times control, or 50-80 seconds.

Heparin modified as taught herein to become 2-O desulfated heparin can provide these many anti-inflammatory benefits with the advantage of greatly reduced anticoagulant activity.

Many modifications and other embodiments of the invention will come to mind to one skilled in the art to which this invention pertains having the benefit of the teachings presented in the foregoing descriptions and the associated drawings. Therefore, it is to be understood that the invention is not to be limited to the specific embodiments disclosed and that modifications and other embodiments are intended to be included, within the scope of the appended claims. Although specific terms are employed herein, they are used in a generic and descriptive sense only and not for purposes of limitation.

What is claimed is:

1. A method for producing an anticoagulant and antithrombotic heparin with enhanced anti-inflammatory activity by mixing 5 to 50 parts by weight of modified heparin demonstrating reduced anticoagulant function with 0 to 1 parts by weight of unmodified heparin to produce an anticoagulant and antithrombotic heparin that both prolongs parameters of coagulation as an anticoagulant and reduces inflammation.

2. The method according to claim 1 wherein said modified, reduced anticoagulant heparin is mixed with unmodified heparin starting material from which it is made in a ratio (weight/weight) of 5 to 50 parts reduced anticoagulant heparin to 0 to 1 part of unmodified, fully anticoagulant heparin.

3. The method according to claim 1 wherein said modified heparin with reduced anticoagulant activity is 2-O desulfated heparin.

4. The method according to claim 1 wherein said modified heparin with reduced anticoagulant function is a 2-O, 3-O desulfated heparin.

5. The method according to claim 3 wherein the 2-O desulfated heparin is made by the process comprising alkalinizing a solution containing heparin to pH 13 or greater.

6. The method according to claim 5, further compromising after the heparin is alkalinized, lyophilizing the alkaline heparin solution.

7. The method according to claim 5, wherein the solution is alkalinized with sodium hydroxide.

8. The method according to claim 1 wherein said modified heparin is produced from porcine intestinal heparin.

9. The method according to claim 1 wherein said modified heparin is produced from bovine lung heparin.

10. The method according to claim 1 wherein said modified heparin is produced from a low molecular weight heparin.

11. The method according to claim 1 wherein said modified heparin with reduced anticoagulant activity is an N-desulfated heparin.

12. The method according to claim 1 wherein said modified heparin with reduced anticoagulant activity is an N-desulfated, N-reacetylated heparin.

13. The method according to claim 1 wherein said modified heparin with reduced anticoagulant activity is a 6-O desulfated heparin.

14. The method according to claim 1 wherein said modified heparin with reduced anticoagulant activity is a fully or partially decarboxylated heparin.

15. The method according to claim 1 wherein said modified heparin with reduced anticoagulant activity is a periodate oxidized heparin.

16. The method according to claim 1 wherein said mixture of reduced anticoagulant heparin and unmodified heparin is administered intravenously.

17. The method according to claim 1 wherein said mixture of reduced anticoagulant heparin and unmodified heparin is administered intravenously in a loading dose of 2.5 to 45 mg/kg, followed by a constant infusion of 0.5 to 9.0 mg/kg/hour.

18. The method according to claim 1 wherein said mixture of reduced anticoagulant heparin and unmodified heparin is administered subcutaneously.

19. The method according to claim 1 wherein said mixture of reduced anticoagulant heparin and unmodified heparin is administered by inhalation.

20. The method according to claim 1 wherein said mixture of reduced anticoagulant heparin and unmodified heparin is administered orally.

21. The method according to claim 1 wherein the mixture of reduced anticoagulant heparin and unmodified heparin is administered rectally.

22. The method according to claim 1 for simultaneously anticoagulating a patient and treating inflammation in adult respiratory distress syndrome, ischemia-reperfusion syndromes, myocardial infarction, stroke, neurologic transient ischemic attacks, atherosclerosis, atherosclerotic vascular disease, acute coronary syndromes, diabetic vascular disease, sepsis, septic shock, disseminated intravascular coagulation, pulmonary embolism, deep vein thrombosis, inflammatory bowel disease, ulcerative colitis, portal vein thrombosis, renal vein thrombosis, thrombosis of the brain venous sinuses, glomerulonephritis, wounds, sickle cell disease, or cutaneous burns.

23. A medicament for treating inflammation in a patient simultaneously in need of anticoagulant or antithrombotic therapy that is produced by mixing 5 to 50 parts by weight of modified heparin with reduced anticoagulant function with 0 to 1 parts by weight of unmodified heparin to produce an anticoagulant and antithrombotic heparin that both prolongs parameters of coagulation as an anticoagulant and reduces inflammation.

24. The medicament according to claim 23 wherein said modified, reduced anticoagulant heparin is mixed with unmodified heparin starting material from which it is made in a ratio (weight/weight) of 5 to 50 parts reduced anticoagulant heparin to 0 to 1 part of unmodified, fully anticoagulant heparin.

25. The medicament according to claim 23 wherein said modified heparin with reduced anticoagulant activity is 2-O desulfated heparin.

26. The medicament according to claim 23 wherein said modified heparin with reduced anticoagulant function is a 2-O, 3-O desulfated heparin.

27. The medicament according to claim 25 wherein the 2-O desulfated heparin is made by the process comprising alkalinizing a solution containing heparin to pH 13 or greater.

28. The medicament according to claim 25, further compromising after the heparin is alkalinized, lyophilizing the alkaline heparin solution.

29. The medicament according to claim 25, wherein the solution is alkalinized with sodium hydroxide.

30. The medicament according to claim 23 wherein said modified heparin is produced from porcine intestinal heparin.

31. The medicament according to claim 23 wherein said modified heparin is produced from bovine lung heparin.

32. The medicament according to claim 23 wherein said modified heparin is produced from a low molecular weight heparin.

33. The medicament according to claim 23 wherein said modified heparin with reduced anticoagulant activity is an N-desulfated heparin.

34. The medicament according to claim 23 wherein said modified heparin with reduced anticoagulant activity is an N-desulfated, N-reacetylated heparin.

35. The medicament according to claim 23 wherein said modified heparin with reduced anticoagulant activity is a 6-O desulfated heparin.

36. The medicament according to claim 23 wherein said modified heparin with reduced anticoagulant activity is a fully or partially decarboxylated heparin.

37. The medicament according to claim 23 wherein said modified heparin with reduced anticoagulant activity is a periodate oxidized heparin.

38. The medicament according to claim 23 wherein the mixture of reduced anticoagulant heparin and unmodified heparin is administered intravenously.

39. The medicament according to claim 23 wherein the mixture of reduced anticoagulant heparin and unmodified heparin is administered intravenously in a loading dose of 2.5 to 45 mg/kg, followed by a constant infusion of 0.5 to 9.0 mg/kg/hour.

40. The medicament according to claim 23 wherein the mixture of reduced anticoagulant heparin and unmodified heparin is administered subcutaneously.

41. The medicament according to claim 23 wherein the mixture of reduced anticoagulant heparin and unmodified heparin is administered by inhalation.

42. The medicament according to claim 23 wherein the mixture of reduced anticoagulant heparin and unmodified heparin is administered orally.

43. The medicament according to claim 23 wherein the mixture of reduced anticoagulant heparin and unmodified heparin is administered rectally.

44. The medicament according to claim 23 for simultaneously anticoagulating a patient and treating inflammation in adult respiratory distress syndrome, ischemia-reperfusion syndromes, myocardial infarction, stroke, neurologic transient ischemic attacks, atherosclerosis, atherosclerotic vascular disease, acute coronary syndromes, diabetic vascular disease, sepsis, septic shock, disseminated intravascular coagulation, pulmonary embolism, deep vein thrombosis, inflammatory bowel disease, ulcerative colitis, portal vein thrombosis, renal vein thrombosis, thrombosis of the brain venous sinuses, glomerulonephritis, wounds, sickle cell disease, or cutaneous burns.
